(19) United States
(12) Patent Application Publication
(10) Pub. No.: US 2022/0220373 A1
(43) Pub. Date: Jul. 14, 2022

(54) FLUORESCENT PROBES FOR MONOACYLGLYCEROL LIPASE (MAGL)

(71) Applicant: Hoffmann-La Roche Inc., Little Falls, NJ (US)

(72) Inventors: Joerg BENZ, Rheinfelden (DE); Thais GAZZI, Berlin (DE); Luca GOBBI, Buus (CH); Uwe GRETHER, Efringen-Kirchen (DE); Benoit HORNSPERGER, Altkirch (FR); Carsten KROLL, Basel (CH); Bernd KUHN, Reinach (CH); Yelena MOSTINSKI, Berlin (DE); Marc NAZARE, Berlin (DE); Fionn O'HARA, Basel (CH); Hans RICHTER, Grenzach-Wyhlen (DE)

(73) Assignee: Hoffmann-La Roche Inc., Little Falls, NJ (US)

(21) Appl. No.: 17/700,987

(22) Filed: Mar. 22, 2022

Related U.S. Application Data
(63) Continuation of application No. PCT/EP2020/076345, filed on Sep. 22, 2020.

(30) Foreign Application Priority Data
Sep. 24, 2019 (EP) 19199218.9

(51) Int. Cl. C09K 11/06; A61K 49/00; G01N 33/58; C07D 498/04; C07F 5/02; C09B 57/00

(52) U.S. Cl. CPC C09K 11/06; A61K 49/0021; G01N 33/582; C07D 498/04; C09K 2211/1014; C09B 57/00; G01N 2333/92; C09K 2211/1022; C09K 2211/1007; C07F 5/027

(57) ABSTRACT

The invention provides fluorescent probes having the general formula (I)

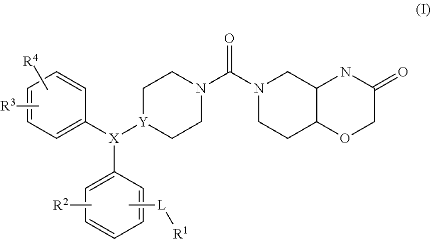

wherein X, Y, L and R¹ to R⁴ are as described herein, compositions including the compounds, processes of manufacturing the compounds and methods of using the compounds.

### CROSS REFERENCE TO RELATED APPLICATIONS

This application is a continuation of International Application No. PCT/EP2020/076345, filed on Sep. 22, 2020, which claims the benefit of EP Application No. 19199218.9, filed on Sep. 24, 2019, the disclosures of which are incorporated herein by reference in their entireties.

### FIELD OF THE INVENTION

The present invention relates to organic compounds useful as fluorescent probes for monoacylglycerol lipase (MAGL).

### BACKGROUND OF THE INVENTION

Fluorescent imaging probes have emerged as high resolution tools to investigate localization, e.g. expression levels and protein distribution in health and disease, structure, dynamics and function of proteins in living cells (L. A. Stoddart, L. E. Kilpatrick, S. J. Briddon, S. J. Hill, Neuropharmacology 2015, 98, 48-57). Such probes can e.g. be applied in flow cytometry fluorescence-activated cell sorting (FACS) experiments or cellular trafficking studies using confocal live cell imaging. Furthermore, fluorescent imaging probes allow for real-time monitoring of ligand-receptor interactions and protein visualization with high spatiotemporal precision (A. J. Vemall, S. J. Hill, B. Kellam, Br. J. Pharmacol. 2014, 171, 1073-1084; C. Iliopoulos-Tsoutsouvas, R. N. Kulkami, A. Makriyannis, S. P. Nikas, Expert Opin. Drug Discov. 2018, 13, 933-947). In addition, such probes offer the potential for generating equilibrium and kinetic binding data in a high-throughput fashion, without handling radioactive material using e.g. time-resolved fluorescence resonance energy transfer (TR-FRET). Fluorescent imaging probes can also be useful to support the translation of preclinical pharmacological animal data to clinics and can be applied for dose selection in humans. They can e.g. be used as markers of target engagement via the generation of ex vivo quantitative receptor binding data in whole blood. Depending on the respective application, a fluorescent imaging probe needs to match specific criteria, including affinity, selectivity and specificity for the respective target, favorable photophysical properties, and applicability across distinct techniques and cell types.

### SUMMARY OF THE INVENTION

In a first aspect (A1), the present invention provides compounds of Formula (I)

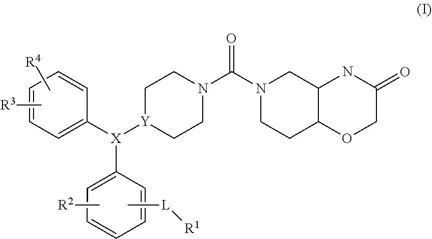

or pharmaceutically acceptable salts thereof, wherein X, Y, L and R¹ to R⁴ are as defined herein.

In a further aspect, the present invention provides a process of manufacturing the compounds of formula (I) as described herein, comprising:
(a) reacting a first amine 4a,5,6,7,8,8a-hexahydro-4H-pyrido[4,3-b][1,4]oxazin-3-one (2),

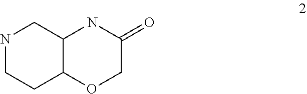 [SMILES: O=C1COC2CCNCC2N1]

with a second amine 1, wherein R², R³, R⁴, L and PG are as defined herein

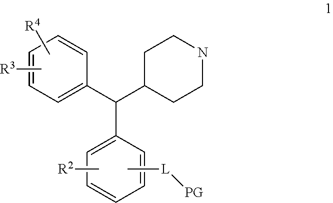

in the presence of a base and a urea forming reagent,
to form a compound of formula 3, wherein R², R³, R⁴, L and PG are as defined herein

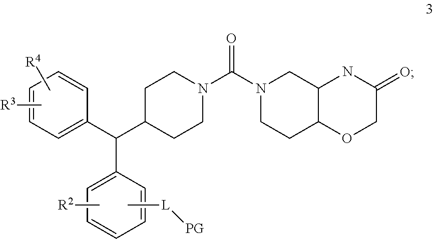

or
(b) reacting a first amine 4a,5,6,7,8,8a-hexahydro-4H-pyrido[4,3-b][1,4]oxazin-3-one (2),

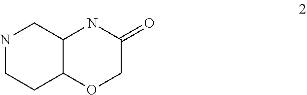 [SMILES: O=C1COC2CCNCC2N1]

with a second amine 1a, wherein R¹, R², R³, R⁴, and L are as defined herein

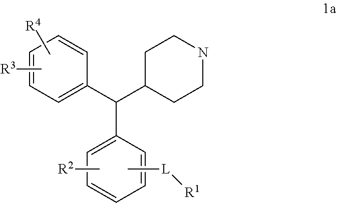

in the presence of a base and a urea forming reagent,
to form said compound of formula (I).

In a further aspect, the present invention provides a compound of formula (I) as described herein, when manufactured according to the processes described herein.

In a further aspect, the present invention provides a compound selected from:
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-(2-aminoethoxy)propanamide;
tert-butyl N-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethyl]carbamate;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-(2-aminoethoxy)ethoxy]propanamide;
tert-butyl N-[2-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethoxy]ethyl]carbamate;
(4aR,8aS)-6-[4-[[3-(2-aminoethoxy)phenyl]-phenyl-methyl]piperidine-1-carbonyl]-4,4a,5,7,8,8a-hexahydropyrido[4,3-b][1,4]oxazin-3-one;
tert-butyl N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-(2-aminoethoxy)ethoxy]ethoxy]propanamide; and
tert-butyl N-[2-[2-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethoxy]ethoxy]ethyl]carbamate;

as well as its use for the preparation of the fluorescent probes of the invention.

In a further aspect, the present invention provides a method of studying monoacylglycerol lipase (MAGL) occupancy, comprising contacting MAGL with a compound of formula (I) described herein.

In a further aspect, the present invention provides a method of diagnostic imaging of monoacylglycerol lipase (MAGL) in a mammal, comprising contacting MAGL with a compound of formula (I) described herein.

In a further aspect, the present invention provides a method of generating monoacylglycerol lipase (MAGL) equilibrium and kinetic binding data, comprising contacting MAGL with a compound of formula (I) described herein.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 12 carbon atoms. In some preferred embodiments, the alkyl group contains 1 to 6 carbon atoms, e.g., 1, 2, 3, 4, 5, or 6 carbon atoms (“C₁-C₆-alkyl”). In other embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. A particularly preferred, yet non-limiting example of alkyl is methyl.

The term “alkoxy” refers to an alkyl group, as previously defined, attached to the parent molecular moiety via an oxygen atom. Unless otherwise specified, the alkoxy group contains 1 to 12 carbon atoms. In some preferred embodiments, the alkoxy group contains 1 to 6 carbon atoms (“C₁-C₆-alkoxy”). In other embodiments, the alkoxy group contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Particularly preferred, yet non-limiting examples of haloalkyl are trifluoromethyl and trifluoroethyl.

The term “haloalkoxy” refers to an alkoxy group, wherein at least one of the hydrogen atoms of the alkoxy group has been replaced by a halogen atom, preferably fluoro. Preferably, “haloalkoxy” refers to an alkoxy group wherein 1, 2 or 3 hydrogen atoms of the alkoxy group have been replaced by a halogen atom, most preferably fluoro. A particularly preferred, yet non-limiting example of haloalkoxy is trifluoromethoxy (—OCF₃).

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are hydrochloride salts.

The term “protective group” (PG) denotes the group which selectively blocks a reactive site in a multifunctional compound such that a chemical reaction can be carried out selectively at another unprotected reactive site in the meaning conventionally associated with it in synthetic chemistry. Protective groups can be removed at the appropriate point. Exemplary protective groups are amino-protective groups, carboxy-protective groups or hydroxy-protective groups. Particular protective groups are the tert-butoxycarbonyl (Boc), benzyloxycarbonyl (Cbz), fluorenylmethoxycarbonyl (Fmoc) and benzyl (Bn). Further particular protective groups are the tert-butoxycarbonyl (Boc) and the fluorenylmethoxycarbonyl (Fmoc). More particular protective group is the tert-butoxycarbonyl (Boc). Exemplary protective groups and their application in organic synthesis are described, for example, in “Protective Groups in Organic Chemistry” by T. W. Greene and P. G. M. Wutts, 5th Ed., 2014, John Wiley & Sons, N.Y.

The term “urea forming reagent” refers to a chemical compound that is able to render a first amine to a species that will react with a second amine, thereby forming an urea derivative. Non-limiting examples of a urea forming reagent include bis(trichloromethyl) carbonate, phosgene, trichloromethyl chloroformate, (4-nitrophenyl)carbonate and 1,1′-carbonyldiimidazole. The urea forming reagents described in G. Sartori et al., Green Chemistry 2000, 2, 140 are incorporated herein by reference.

The compounds of formula (I) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The abbreviation “MAGL” refers to the enzyme monoacylglycerol lipase. The terms “MAGL” and “monoacylglycerol lipase” are used herein interchangeably.

### Compounds of the Invention

In a first aspect (A1), the present invention provides compounds of Formula (I)

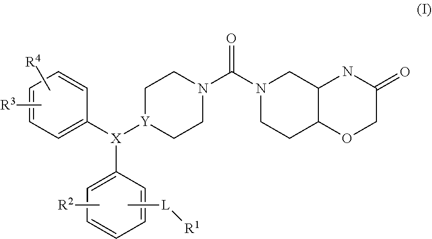

or pharmaceutically acceptable salts thereof, wherein:
L is a linker;
R¹ is a fluorescent label;
R², R³, and R⁴ are each independently selected from hydrogen, halogen, C₁-C₆-alkyl, halo-C₁-C₆-alkyl, C₁-C₆-alkoxy and halo-C₁-C₆-alkoxy; and
X and Y are both CH; or
X and Y taken together form a double bond (C═C).

In one embodiment, said compound of formula (I) is a compound of formula (Ia)

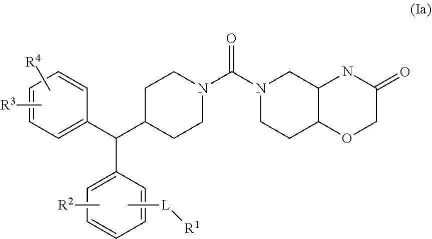

wherein L and R¹ to R⁴ are as defined herein.

The invention also provides the following enumerated Embodiments (E) of the first aspect (A1) of the invention:
E1. The compound of formula (I) according to A1, or a pharmaceutically acceptable salt thereof, wherein
   L is a linker selected from

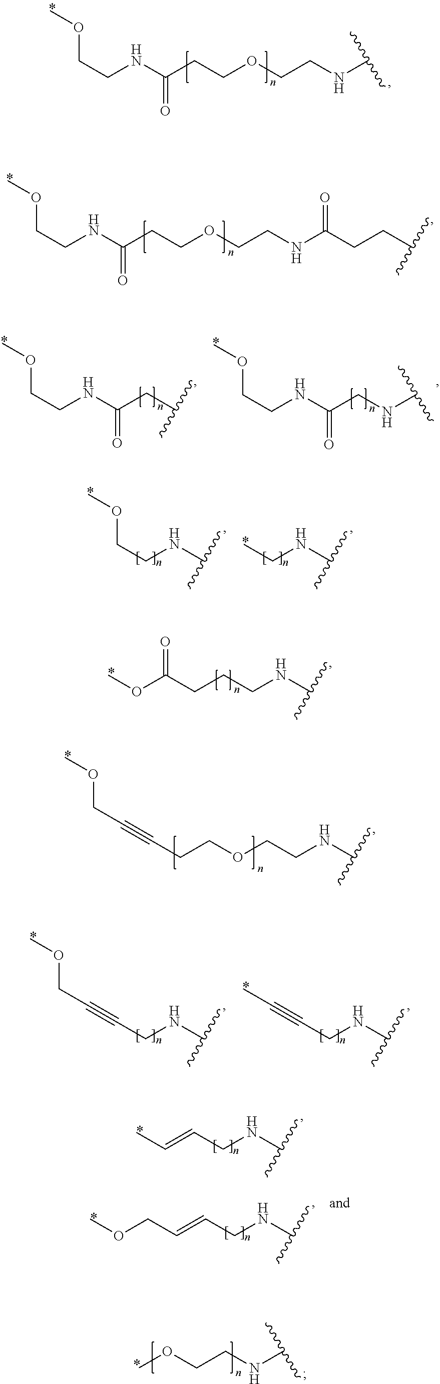

wherein
each occurrence of n is independently an integer selected from 1, 2, 3, 4, 5, 6, and 7;
a wavy line indicates the point of attachment to R¹; and
an asterisk indicates the point of attachment to the rest of formula (I);
R¹ is a fluorescent label selected from

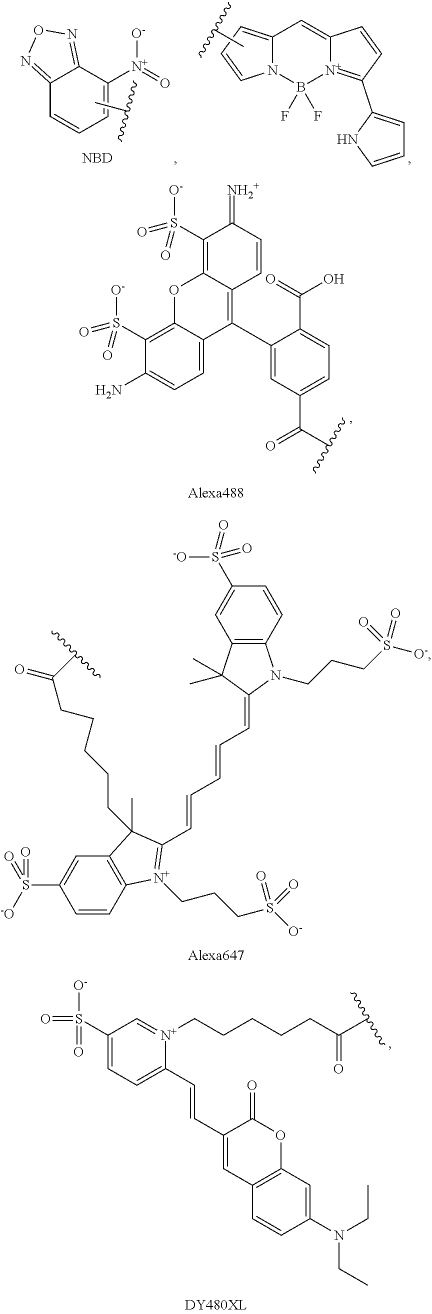

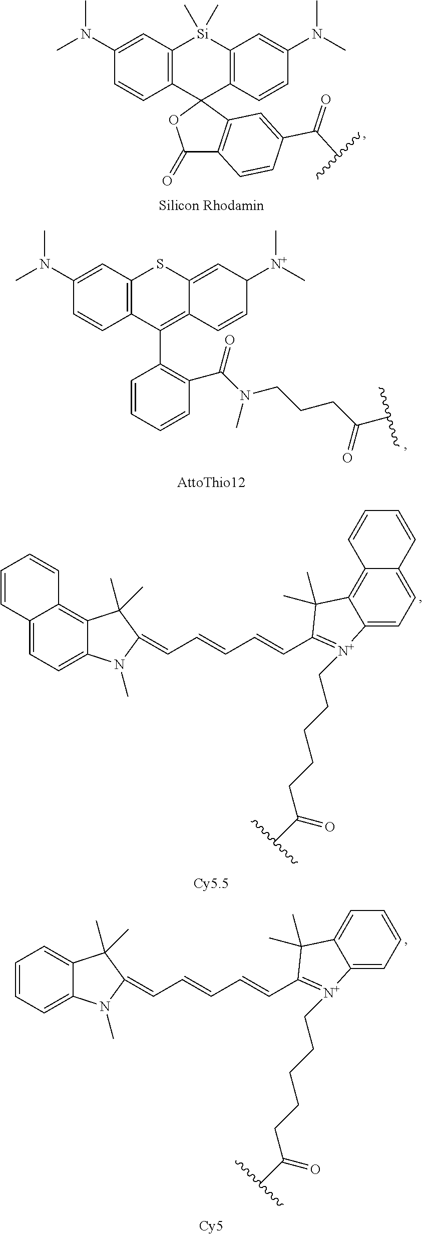

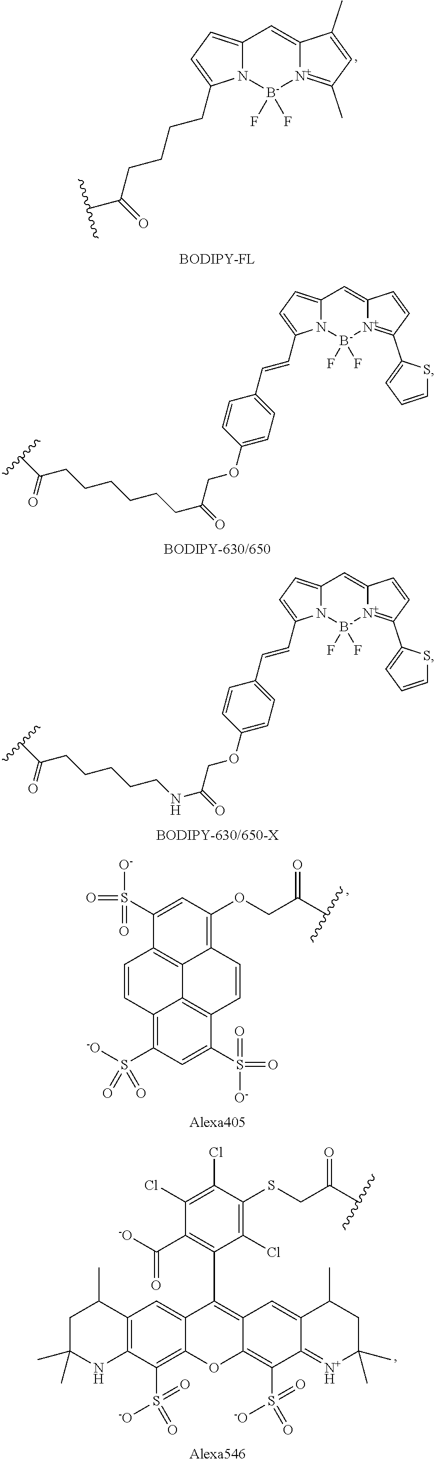

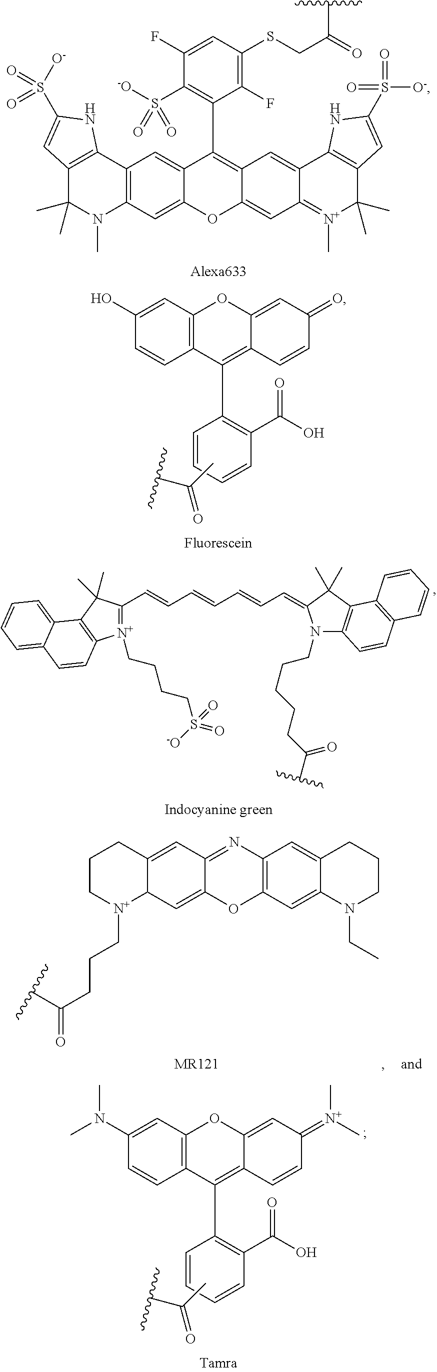

wherein a wavy line indicates the point of attachment to the linker L;
R², R³, and R⁴ are each independently selected from hydrogen, halogen, C₁-C₆-alkyl, halo-C₁-C₆-alkyl, C₁-C₆-alkoxy and halo-C₁-C₆-alkoxy; and
X and Y are both CH; or
X and Y taken together form a double bond (C═C).
E2. The compound of formula (I) according to A1, or a pharmaceutically acceptable salt thereof, wherein the linker L is selected from

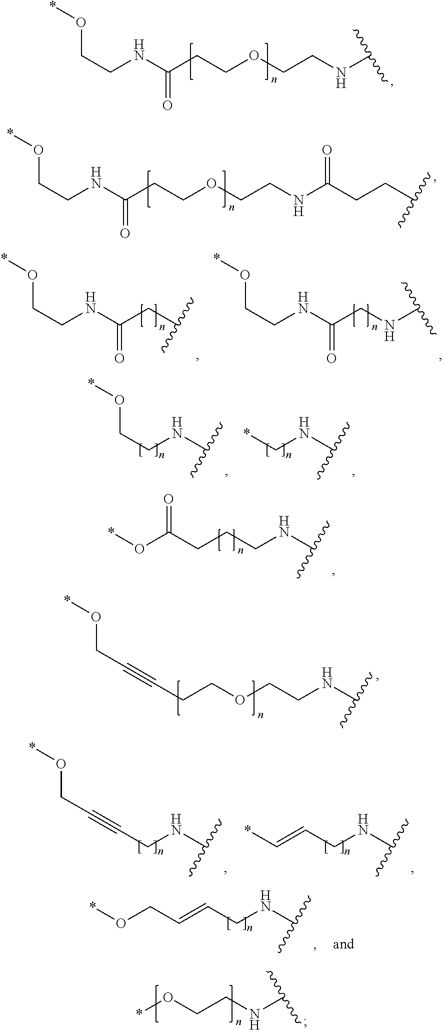

wherein
each occurrence of n is independently an integer selected from 1, 2, 3, 4, 5, 6, and 7;
a wavy line indicates the point of attachment to the fluorescent label R¹; and
an asterisk indicates the point of attachment to the rest of formula (I).
E3. The compound of formula (I) according to A1 and E1, or a pharmaceutically acceptable salt thereof, wherein the linker L is selected from

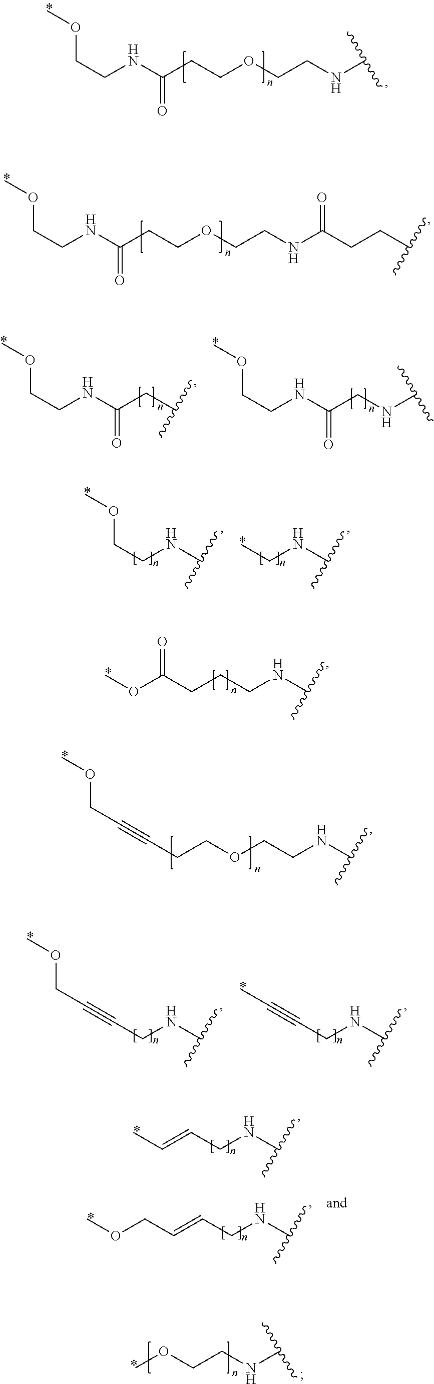

wherein
each occurrence of n is independently an integer selected from 1, 2, 3, 4, 5, 6, and 7;
a wavy line indicates the point of attachment to the fluorescent label R¹; and
an asterisk indicates the point of attachment to the rest of formula (I).
E4. The compound of formula (I) according to A1 and E1, or a pharmaceutically acceptable salt thereof, wherein the linker L is selected from

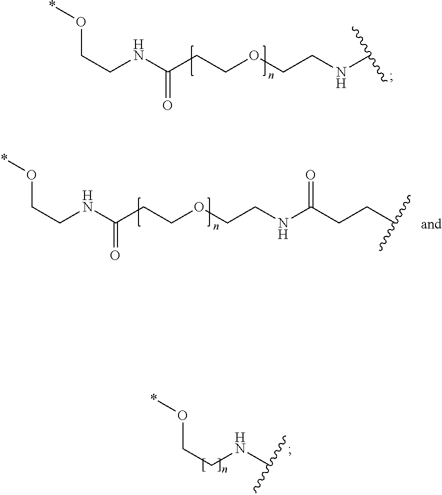

wherein
each occurrence of n is independently an integer selected from 1, 2, and 3;
a wavy line indicates the point of attachment to the fluorescent label R¹; and
an asterisk indicates the point of attachment to the rest of formula (I).
E5. The compound of formula (I) according to A1 and E1, or a pharmaceutically acceptable salt thereof, wherein the linker L is selected from

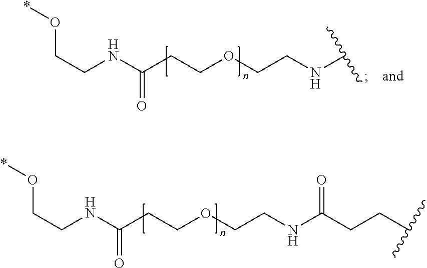

wherein
each occurrence of n is independently an integer selected from 1, 2, and 3;
a wavy line indicates the point of attachment to the fluorescent label R¹; and
an asterisk indicates the point of attachment to the rest of formula (I).
E6. The compound of formula (I) according to A1 and E1, or a pharmaceutically acceptable salt thereof, wherein the linker L is selected from

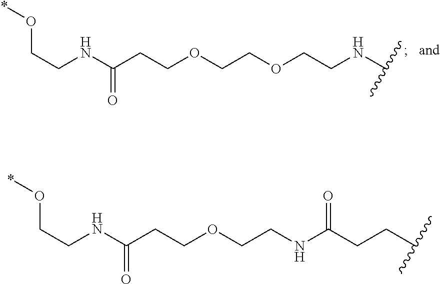 [SMILES: *NCCOCCOCCC(=O)NCCO* ; *CCC(=O)NCCOCCC(=O)NCCO*]

wherein
a wavy line indicates the point of attachment to the fluorescent label R¹; and
an asterisk indicates the point of attachment to the rest of formula (I).
E7. The compound of formula (I) according to A1 and E1, or a pharmaceutically acceptable salt thereof, wherein the linker L is selected from

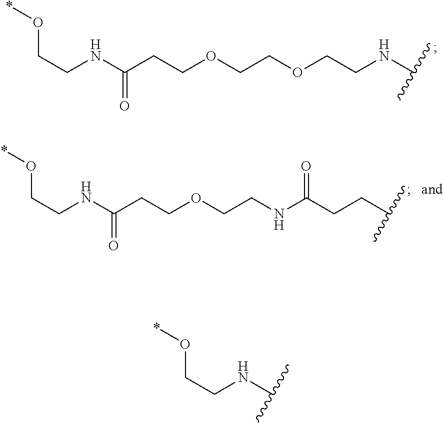 [SMILES: *NCCOCCOCCC(=O)NCCO* ; *CCC(=O)NCCOCCC(=O)NCCO* ; *NCCO*]

wherein
a wavy line indicates the point of attachment to the fluorescent label R¹; and
an asterisk indicates the point of attachment to the rest of formula (I).
E8. The compound of formula (I) according to any one of A1 and E2 to E7, or a pharmaceutically acceptable salt thereof, wherein the fluorescent label R¹ is selected from

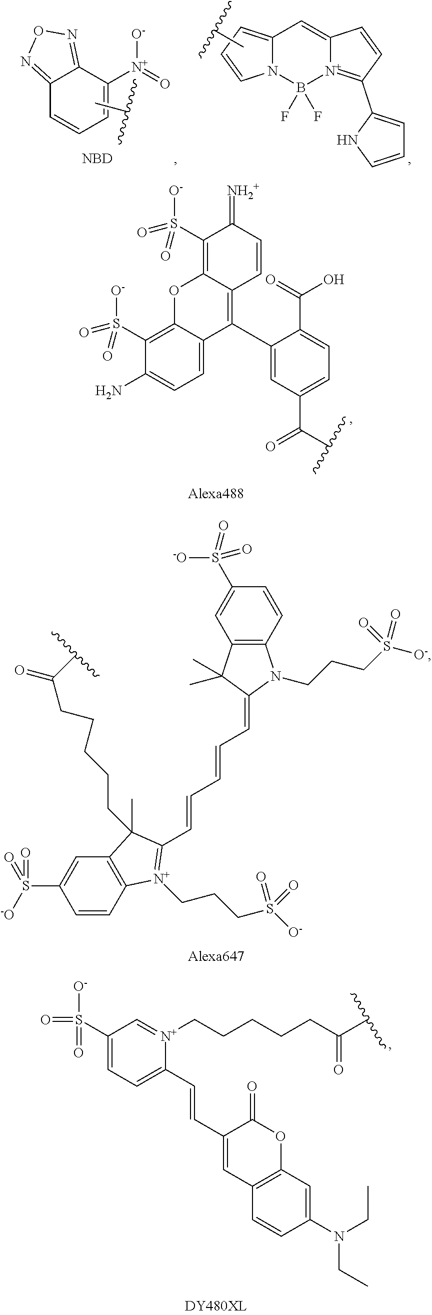

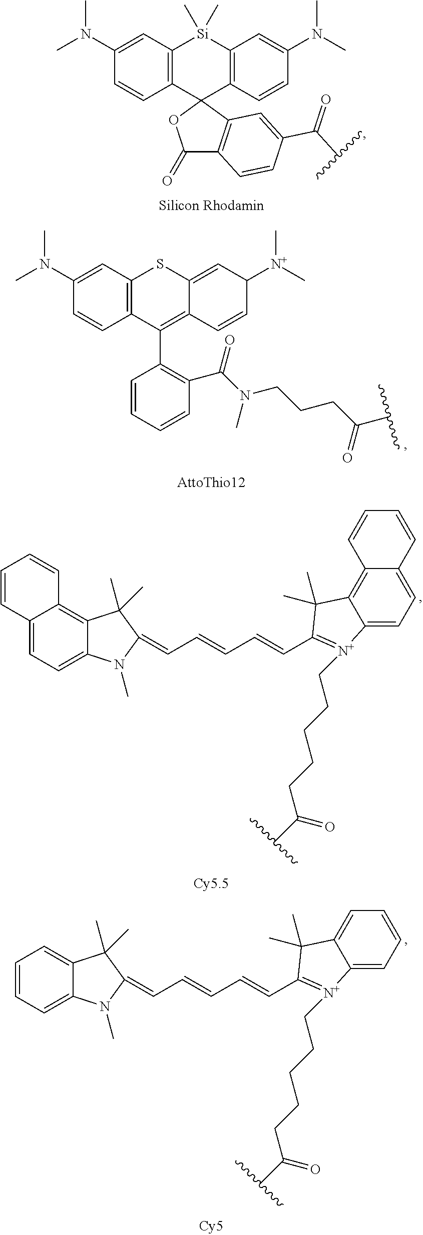

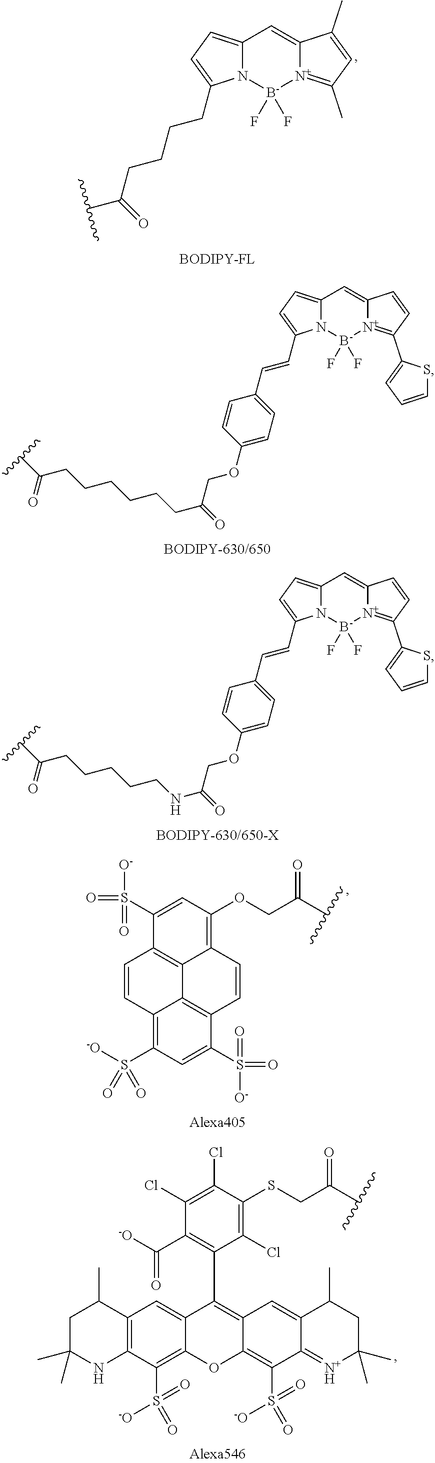

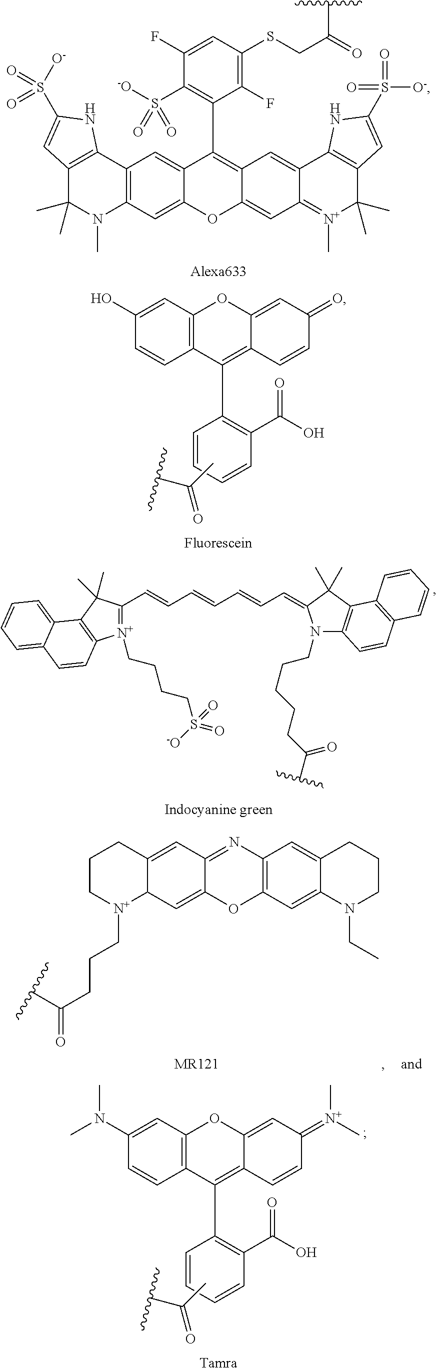

wherein a wavy line indicates the point of attachment to the linker L.
E9. The compound of formula (I) according to any one of A1 and E1 to E7, or a pharmaceutically acceptable salt thereof, wherein the fluorescent label R¹ is selected from

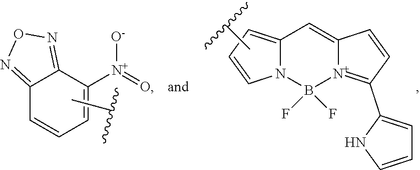

wherein a wavy line indicates the point of attachment to the linker L.
E10. The compound of formula (I) according to any one of A1 and E1 to E7, or a pharmaceutically acceptable salt thereof, wherein the fluorescent label R¹ is selected from

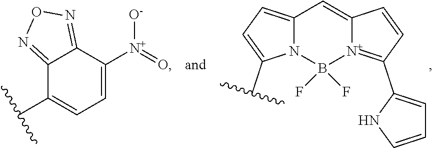

wherein a wavy line indicates the point of attachment to the linker L.
E11. The compound of formula (I) according to any one of A1 and E1 to E10, or a pharmaceutically acceptable salt thereof, wherein R² is hydrogen.
E12. The compound of formula (I) according to any one of A1 and E1 to E11, or a pharmaceutically acceptable salt thereof, wherein R³ is hydrogen.
E13. The compound of formula (I) according to any one of A1 and E1 to E12, or a pharmaceutically acceptable salt thereof, wherein R⁴ is hydrogen.
E14. The compound of formula (I) according to A1, or a pharmaceutically acceptable salt thereof, wherein the compound of formula (I) is a compound of formula (II)

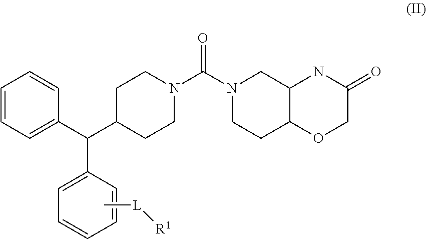

wherein the linker L is selected from

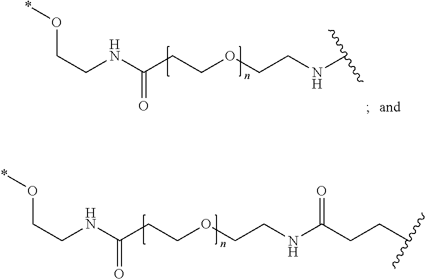

wherein
each occurrence of n is independently an integer selected from 1, 2, and 3;
a wavy line indicates the point of attachment to the fluorescent label R¹; and
an asterisk indicates the point of attachment to the rest of formula (I);
and wherein the fluorescent label R¹ is selected from

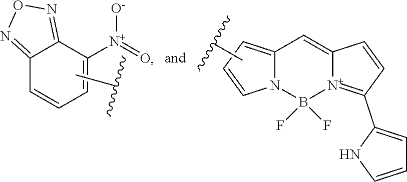

wherein a wavy line indicates the point of attachment to the linker L.
E15. The compound of formula (I) according to A1, or a pharmaceutically acceptable salt thereof, wherein the compound of formula (I) is a compound of formula (II) or (III)

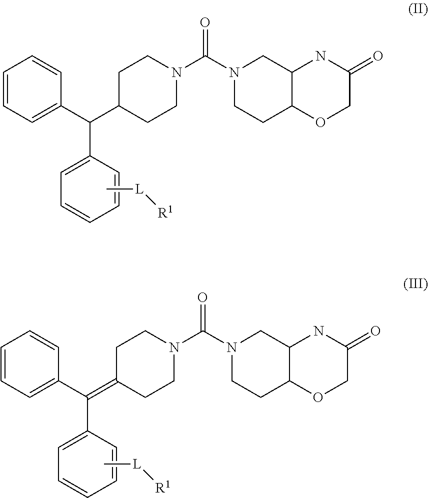

wherein the linker L is selected from

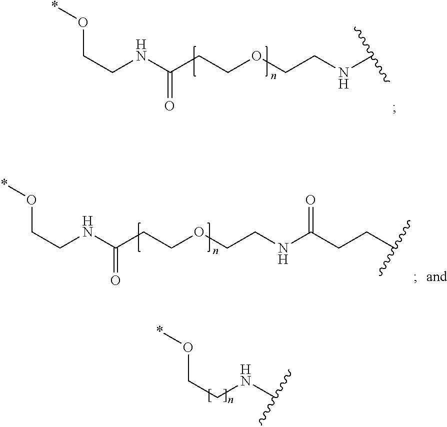

wherein
each occurrence of n is independently an integer selected from 1, 2, and 3;
a wavy line indicates the point of attachment to the fluorescent label R¹; and
an asterisk indicates the point of attachment to the rest of formula (I);
and wherein the fluorescent label R¹ is selected from

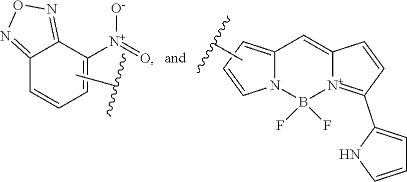

wherein a wavy line indicates the point of attachment to the linker L.
E16. The compound of formula (I) according to A1, or a pharmaceutically acceptable salt thereof, wherein the compound of formula (I) is a compound of formula (II)

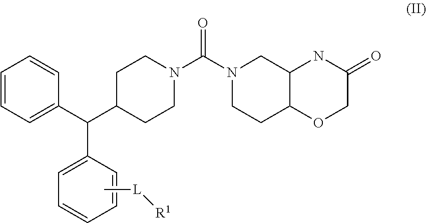

wherein the linker L is selected from

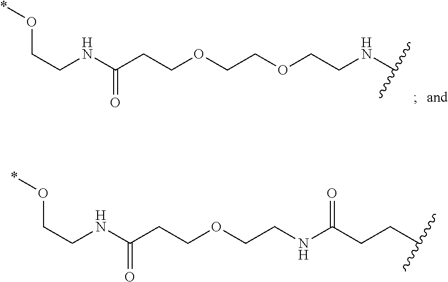

wherein
each occurrence of n is independently an integer selected from 1, 2, and 3;
a wavy line indicates the point of attachment to the fluorescent label R¹; and
an asterisk indicates the point of attachment to the rest of formula (I);
and wherein the fluorescent label R¹ is selected from

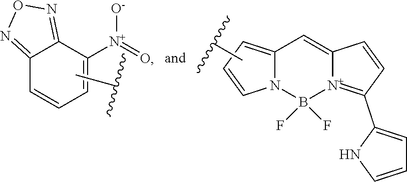

wherein a wavy line indicates the point of attachment to the linker L.
E17. The compound of formula (I) according to A1, or a pharmaceutically acceptable salt thereof, wherein the compound of formula (I) is a compound of formula (II) or (III)

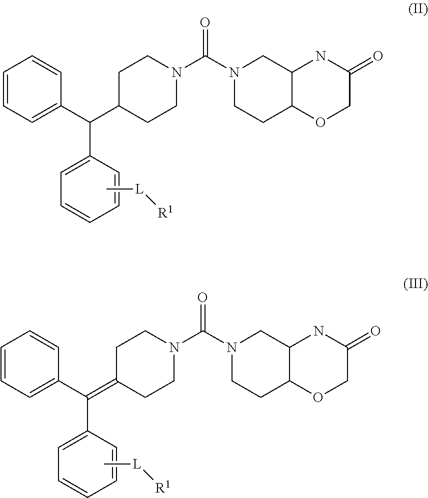

wherein the linker L is selected from

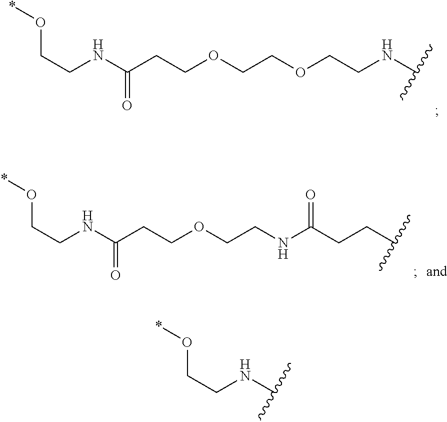 [SMILES: *NCCOCCOCCC(=O)NCCO* ; *CCC(=O)NCCOCCC(=O)NCCO* ; *NCCO*]

wherein
a wavy line indicates the point of attachment to the fluorescent label R¹; and
an asterisk indicates the point of attachment to the rest of formula (I);
and wherein the fluorescent label R¹ is selected from

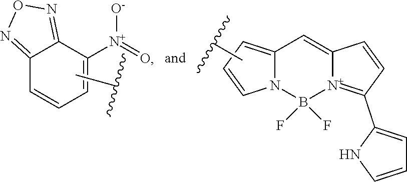

wherein a wavy line indicates the point of attachment to the linker L.
E18. The compound of formula (I) according to any one of A1 and E1 to E17, or a pharmaceutically acceptable salt thereof, wherein the compound of formula (I) is selected from:
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[(4-nitro-2,1,3-benzoxadiazol-7-yl)amino]ethoxy]propanamide;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[(4-nitro-2,1,3-benzoxadiazol-7-yl)amino]ethoxy]propanamide;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-[(4-nitro-2,1,3-benzoxadiazol-7-yl)amino]ethoxy]ethoxy]propanamide;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-[(4-nitro-2,1,3-benzoxadiazol-7-yl)amino]ethoxy]ethoxy]propanamide;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-[(4-nitro-2,1,3-benzoxadiazol-7-yl)amino]ethoxy]ethoxy]propanamide;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[(4-nitro-2,1,3-benzoxadiazol-7-yl)amino]ethoxy]propanamide;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-[2-[(4-nitro-2,1,3-benzoxadiazol-7-yl)amino]ethoxy]ethoxy]ethoxy]propanamide;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-[2-[(4-nitro-2,1,3-benzoxadiazol-7-yl)amino]ethoxy]ethoxy]ethoxy]propanamide;
N-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethyl]-3-[2,2-difluoro-12-(1H-pyrrol-2-yl)-3-aza-1-azonia-2-boranuidatricyclo[7.3.0.03,7]dodeca-1(12),4,6,8,10-pentaen-4-yl]propanamide; and
(4aR,8a5)-6-(4-((3-(2-((7-Nitrobenzo[c][1,2,5]oxadiazol-4-yl)amino)ethoxy)phenyl)(phenyl)methylene)piperidine-1-carbonyl)hexahydro-2H-pyrido[4,3-b][1,4]oxazin-3(4H)-one.
E19. The compound of formula (I) according to any one of A1 and E1 to E17, or a pharmaceutically acceptable salt thereof, wherein the compound of formula (I) is selected from:
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-[(4-nitro-2,1,3-benzoxadiazol-7-yl)amino]ethoxy]ethoxy]propanamide;
N-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethyl]-3-[2,2-difluoro-12-(1H-pyrrol-2-yl)-3-aza-1-azonia-2-boranuidatricyclo[7.3.0.03,7]dodeca-1(12),4,6,8,10-pentaen-4-yl]propanamide.
E20. The compound of formula (I) according to any one of A1 and E1 to E17, or a pharmaceutically acceptable salt thereof, wherein the compound of formula (I) is selected from:
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-[(4-nitro-2,1,3-benzoxadiazol-7-yl)amino]ethoxy]ethoxy]propanamide;
N-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethyl]-3-[2,2-difluoro-12-(1H-pyrrol-2-yl)-3-aza-1-azonia-2-boranuidatricyclo[7.3.0.03,7]dodeca-1(12),4,6,8,10-pentaen-4-yl]propanamide; and
(4aR,8a5)-6-(4-((3-(2-((7-Nitrobenzo[c][1,2,5]oxadiazol-4-yl)amino)ethoxy)phenyl)(phenyl)methylene)piperidine-1-carbonyl)hexahydro-2H-pyrido[4,3-b][1,4]oxazin-3(4H)-one.

The present invention also relates to synthetic intermediates (i.e., unlabeled compounds) that are useful for making the fluorescent probes of the invention.

Enumerated embodiments 21 to 23 provide preferred examples of such synthetic intermediates.
E21. A compound selected from:
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-(2-aminoethoxy)propanamide;
tert-butyl N-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethyl]carbamate;
tert-butyl N-[2-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethoxy]ethyl]carbamate;
(4aR,8a5)-6-[4-[[3-(2-aminoethoxy)phenyl]-phenyl-methyl]piperidine-1-carbonyl]-4,4a,5,7,8,8a-hexahydropyrido[4,3-b][1,4]oxazin-3-one;
tert-butyl N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-(2-aminoethoxy)ethoxy]ethoxy]propanamide;
tert-butyl N-[2-[2-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethoxy]ethoxy]ethyl]carbamate; and
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-(2-aminoethoxy)ethoxy]propanamide.
E22. A compound selected from:
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-(2-aminoethoxy)propanamide;
tert-butyl N-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethyl]carbamate;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-(2-aminoethoxy)ethoxy]propanamide;
tert-butyl N-[2-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethoxy]ethyl]carbamate;
(4aR,8aS)-6-[4-[[3-(2-aminoethoxy)phenyl]-phenyl-methyl]piperidine-1-carbonyl]-4,4a,5,7,8,8a-hexahydropyrido[4,3-b][1,4]oxazin-3-one;
tert-butyl N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-(2-aminoethoxy)ethoxy]ethoxy]propanamide;
tert-butyl N-[2-[2-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethoxy]ethoxy]ethyl]carbamate;
tert-Butyl (2-(3-((1-((4aR,8a5)-3-oxooctahydro-2H-pyrido[4,3-b][1,4]oxazine-6-carbonyl)piperidin-4-ylidene)(phenyl)methyl)phenoxy)ethyl)carbamate;
tert-Butyl (2-(4-((1-((4aR,8a5)-3-oxooctahydro-2H-pyrido[4,3-b][1,4]oxazine-6-carbonyl)piperidin-4-ylidene)(phenyl)methyl)phenoxy)ethyl)carbamate;
tert-Butyl N-[3-[3-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenyl]prop-2-ynyl]carbamate;
tert-Butyl N-[3-[4-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenyl]prop-2-ynyl]carbamate;
tert-Butyl N-[(E)-3-[3-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenyl]allyl]carbamate;
tert-Butyl N-[(E)-3-[4-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenyl]allyl]carbamate;
tert-Butyl N-[2-[3-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate;
tert-Butyl N-[2-[4-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate;
tert-Butyl N-[3-[3-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenyl]propyl]carbamate;
tert-Butyl N-[3-[4-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenyl]propyl]carbamate;
tert-Butyl N-[3-[4-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenyl]prop-2-ynyl]carbamate;
tert-Butyl N-[3-[3-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenyl]prop-2-ynyl]carbamate;
tert-Butyl N-[(E)-3-[4-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenyl]allyl]carbamate;
tert-Butyl N-[(E)-3-[3-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenyl]allyl]carbamate;
tert-Butyl N-[2-[3-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenoxy]ethylamino]-3-oxo-propoxy]ethyl]carbamate;
tert-Butyl N-[3-[4-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenyl]propyl]carbamate;
tert-Butyl N-[3-[3-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenyl]propyl]carbamate.
E23. A compound selected from:
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-(2-aminoethoxy)propanamide;
tert-butyl N-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethyl]carbamate;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-(2-aminoethoxy)ethoxy]propanamide;
tert-butyl N-[2-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethoxy]ethyl]carbamate;
(4aR,8a5)-6-[4-[[3-(2-aminoethoxy)phenyl]-phenyl-methyl]piperidine-1-carbonyl]-4,4a,5,7,8,8a-hexahydropyrido[4,3-b][1,4]oxazin-3-one;
tert-butyl N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-(2-aminoethoxy)ethoxy]ethoxy]propanamide;
tert-butyl N-[2-[2-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethoxy]ethoxy]ethyl]carbamate;
tert-Butyl (2-(3-((1-((4aR,8aS)-3-oxooctahydro-2H-pyrido[4,3-b][1,4]oxazine-6-carbonyl)piperidin-4-ylidene)(phenyl)methyl)phenoxy)ethyl)carbamate; and
tert-Butyl (2-(4-((1-((4aR,8a5)-3-oxooctahydro-2H-pyrido[4,3-b][1,4]oxazine-6-carbonyl)piperidin-4-ylidene)(phenyl)methyl)phenoxy)ethyl)carbamate.
E24. Use of a compound according to any one of E21-E23 for the preparation of a fluorescent probe of formula (I) according to any one of A1 and E1 to E20.

In a particular embodiment, the present invention provides pharmaceutically acceptable salts of the compounds according to formula (I) as described herein. In a further particular embodiment, the present invention provides compounds according to formula (I) as described herein as free bases.

### Processes of Manufacturing

The preparation of compounds of formula (I) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (I) will typically lead to the respective diastereomerically/enantiomerically enriched compounds of formula (I).

A person skilled in the art will acknowledge that in the synthesis of compounds of formula (I)-insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, N.Y. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

The following abbreviations are used in the present text:

AcOH=acetic acid, ACN=acetonitrile, Boc=tert-butyloxycarbonyl, CAS RN=chemical abstracts registration number, Cbz=benzyloxycarbonyl, Cs₂CO₃=cesium carbonate, CO=carbon monoxide, CuCl=copper(I) chloride, CuCN=copper(I) cyanide, CuI=copper(I) iodide, DMAP=4-dimethylaminopyridine, DME=dimethoxyethane, DMEDA=N,N′-dimethylethylenediamine, DMF=N,N-dimethylformamide, DIPEA=N,N-diisopropylethylamine, dppf=1,1 bis(diphenyl phosphino)ferrocene, EDC.HCl=N-(3-dimethylaminopropyl)-N′-ethylcarbodiimide hydrochloride, EI=electron impact, ESI=electrospray ionization, EtOAc=ethyl acetate, EtOH=ethanol, h=hour(s), FA=formic acid, H₂O=water, H₂SO₄=sulfuric acid, Hal=halogen, HATU=1-[bis(dimethylamino)methylene]-1H-1,2,3-triazolo[4,5-b]pyridinium-3-oxide hexafluorophosphate, HBTU=O-benzotriazole-N,N,N′,N′-tetramethyl-uronium-hexafluoro-phosphate, HCl=hydrogen chloride, HOBt=1-hydroxy-1H-benzotriazole; HPLC=high performance liquid chromatography, iPrMgCl=isopropylmagnesium chloride, 12=iodine, IPA=2-propanol, (Ir[dF(CF₃)ppy]₂(dtbpy))PF₆=[4,4′-bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate, ISP=ion spray positive (mode), ISN=ion spray negative (mode), K₂CO₃=potassium carbonate, KHCO₃=potassium bicarbonate, KI=potassium iodide, KOH=potassium hydroxide, K₃PO₄=potassium phosphate tribasic, LiA₁H₄ or LAH=lithium aluminium hydride, LiHMDS=lithium bis(trimethylsilyl)amide, LiOH=lithium hydroxide, MgSO₄=magnesium sulfate, min=minute(s), mL=milliliter, MPLC=medium pressure liquid chromatography, MS=mass spectrum, NaH=sodium hydride, NaHCO₃=sodium hydrogen carbonate, NaNO₂=sodium nitrite, NaOH=sodium hydroxide, Na₂CO₃=sodium carbonate, Na₂SO₄=sodium sulfate, Na₂S₂O₃=sodium thiosulfate, NBS=N-bromosuccinimide, nBuLi=n-butyllithium, NEt₃=triethylamine (TEA), NH₄Cl=ammonium chloride, NiCl₂ glyme=Nickel(II) chloride ethylene glycol dimethyl ether complex, NMP=N-methyl-2-pyrrolidone, OAc=Acetoxy, T₃P=propylphosphonic anhydride, P2O5=phosphorus pentoxide, PE=petroleum ether, PG=protective group, Pd—C=palladium on activated carbon, PdCl₂(dppf)-CH₂Cl₂=1,1′-bis(diphenylphosphino)ferrocene-palladium(II)dichloride dichloromethane complex, Pd₂(dba)₃=tris(dibenzylideneacetone)dipalladium(0), Pd(OAc)₂=palladium(II) acetate, Pd(OH)₂=palladium hydroxide, Pd(PPh₃)₄=tetrakis(triphenylphosphine)palladium(0), PTSA=p-toluenesulfonic acid, R=any group, RT=room temperature, SFC=Supercritical Fluid Chromatography, S—PHOS=2-dicyclohexylphosphino-2′,6′-dimethoxybiphenyl, T3P=propylphosphonic anhydride, TBAI=tetra butyl ammonium iodine, TEA=triethylamine, TFA=trifluroacetic acid, THF=tetrahydrofuran, TMEDA=N,N,N′,N′-tetramethylethylenediamine, ZnCl₂=zinc chloride, Xantphos=4,5-Bis(diphenylphosphino)-9,9-dimethylxanthene.

Compounds of formula I wherein L, n, R¹, R², R³ and R⁴ are as described herein can be synthesized in analogy to literature procedures and/or as depicted for example in Scheme 1.

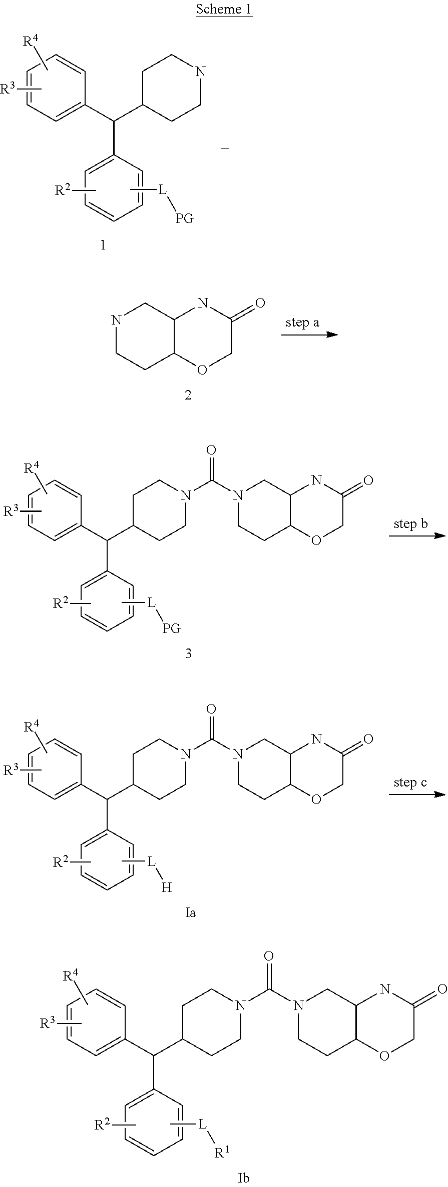

Accordingly, 4a,5,6,7,8,8a-hexahydro-4H-pyrido[4,3-b][1,4]oxazin-3-ones 2 are reacted with intermediates 1 in the presence of a urea forming reagent such as bis(trichloromethyl) carbonate using a suitable base and solvent such as, e.g. sodium bicarbonate in DCM, to give compounds of formula 3 (step a). Further urea forming reagents include but are not limited to phosgene, trichloromethyl chloroformate, (4-nitrophenyl)carbonate or 1,1′-carbonyldiimidazole. Reactions of this type and the use of these reagents are widely described in literature (e.g. G. Sartori et al., Green Chemistry 2000, 2, 140). A person skilled in the art will acknowledge that the order of the addition of the reagents can be important in this type of reactions due to the reactivity and stability of the intermediary formed carbamoyl chlorides, as well as for avoiding formation of undesired symmetrical urea by-products. Piperidine derivatives 1 carry a suitable protective group “PG” such as a Cbz or Boc protective group. Piperidines 1 and 4a,5,6,7,8,8a-hexahydro-4H-pyrido[4,3-b][1,4]oxazin-3-ones 2 are either commercially available or can be prepared according to literature methods or as depicted below.

Removal of the protective group in intermediates 3, applying methods known in the art (e.g., a Boc group using TFA in DCM at temperatures between 0° C. and room temperature, a Cbz group using hydrogen in the presence of a suitable catalyst such as Pd or Pd(OH)₂ on charcoal in a suitable solvent such as MeOH, EtOH, EtOAc or mixtures thereof and as described for example in “Protective Groups in Organic Chemistry” by T. W. Greene and P. G. M. Wuts, 4th Ed., 2006, Wiley N.Y.), furnishes molecules of formula Ia (step b).

Reaction of amines Ia with a fluorescent label containing a carboxylic acid moiety gives compounds of formula Ib (R¹=fluorescent label) (step c). Amide couplings of this type can be accomplished by using one of the well-known coupling reagents such as, DCC, HATU, EDCI, HOBt, TBTU, T3P, etc. and a base like Huenig's base, triethyl amine or DMAP in a suitable solvent solvent like N,N-dimethylformamide, DMA, DCM or dioxane, preferably between 0° C. and room temperature. To form final molecules Ib carrying a nitrobenzofurazan dye, amines Ia can be reacted in a nucleophic aromatic substitution with 4-chloro-7-nitrobenz-2-oxa-1,3-diazole (CAS RN 10199-89-0), e.g. in the presence of a base such as N,N-diisopropylethylamine in a solvent as MeOH, preferentially at ambient temperature (step c).

Optionally, piperidine derivatives 1 in which “PG” is a fluorescent label or tertbutoxycarbonyl can be converted directly to compounds of formula (Ib) according to step a. In case “PG” is tertbutoxycarbonyl final molecules of formula (Ib) with R¹ being equal to tertbutoxycarbonyl can be obtained.

4a,5,6,7,8,8a-hexahydro-4H-pyrido[4,3-b][1,4]oxazin-3-ones 2 may be synthesized as depicted for example in Scheme 2 and/or in analogy to methods described in literature.

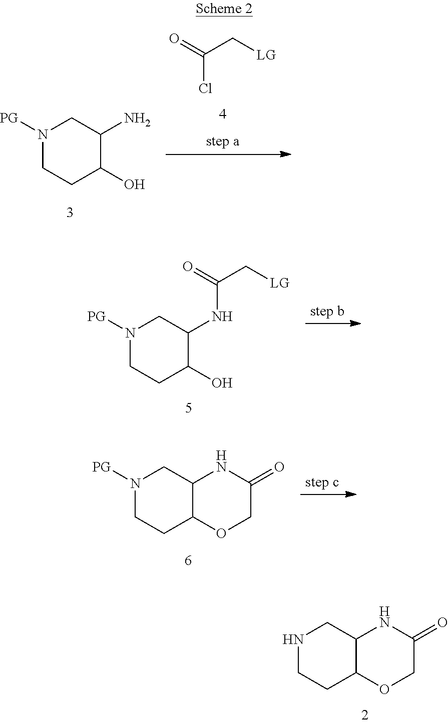

Thus, 3-aminopiperidin-4-ol derivatives 3 in which “PG” signifies a suitable protective group such as a Cbz or Boc protective group can be acylated for example with acyl chlorides 4 in which “LG” signifies a suitable leaving group (e.g., Cl or Br), using a suitable base such as sodium or potassium carbonate, sodium hydroxide or sodium acetate in an appropriate solvent such as THF, water, acetone or mixtures thereof, to provide intermediates 5 (step a). Intermediates 4 are either commercially available or can be prepared according to literature methods. Intermediates 5 can be cyclized to intermediates 6 using methods well known in the art, for example by treatment of 5 with sodium hydride in THF or potassium tert-butoxide in IPA and water (step b). Reactions of that type are described in literature (e.g. Z. Rafinski et al., J. Org. Chem. 2015, 80, 7468; S. Dugar et al., Synthesis 2015, 47(5), 712; WO2005/066187). Removal of the protective group in intermediates 6, applying methods known in the art (e.g., a Boc group using TFA in DCM at temperatures between 0° C. and room temperature, a Cbz group using hydrogen in the presence of a suitable catalyst such as Pd or Pd(OH)₂ on charcoal in a suitable solvent such as MeOH, EtOH, EtOAc or mixtures thereof and as described for example in “Protective Groups in Organic Chemistry” by T. W. Greene and P. G. M. Wuts, 4th Ed., 2006, Wiley N.Y.), furnishes intermediates 2 (step c).

Intermediates 2 can be obtained as mixtures of diastereomers and enantiomers, respectively, or as single stereoisomers depending on whether racemic mixtures or enantiomerically pure forms of cis- or trans-3-aminopiperidin-4-ol derivatives 3 are employed in their syntheses. Intermediates 3 are commercially available and their synthesis has also been described in literature (e.g.

WO2005/066187; WO2011/0059118; WO2016/185279). Optically pure cis-configured intermediates 2 can be obtained for example by chiral separation of commercially available rac-(4aR,8a5)-4a,5,6,7,8,8a-hexahydro-4H-pyrido[4,3-b][1,4]oxazin-3-one (optionally in form of a salt such as, e.g. a hydrochloride salt) using methods known in the art, e.g. by diastereomeric salt crystallization or by chiral chromatography.
Intermediates of type 1 can be prepared by a variety of conditions, which may be exemplified by the general synthetic procedure outlined in Scheme 3.

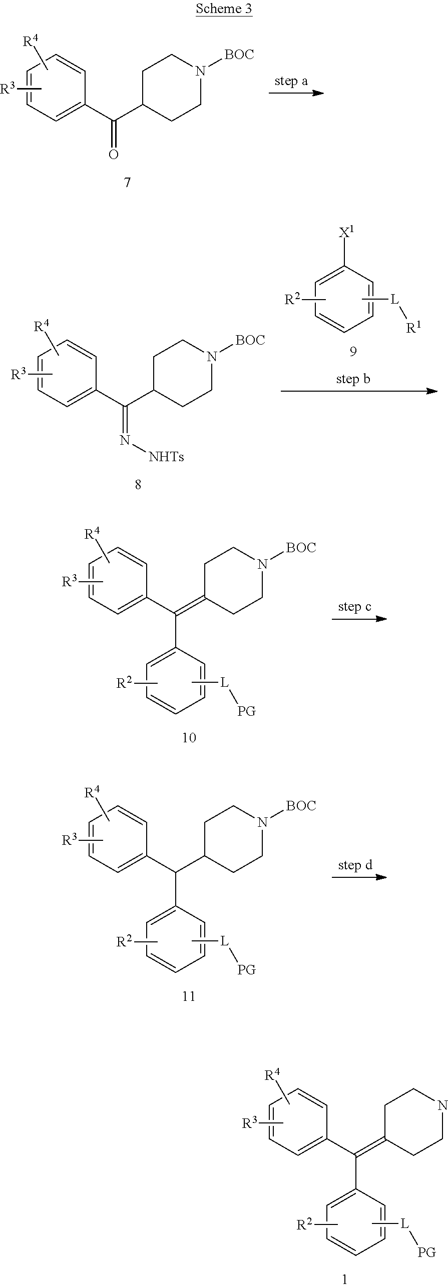

A ketone of formula 7 can be reacted to a N-tosylhydrazone 8, for example using NH₂NHTs in a solvent as 1,4-dioxane preferably by heating to 80° C. (step a). In a further step N-tosylhydrazones of formula 8 are reacted with aryl halides 9, wherein X¹ is selected from the group consisting of Cl, Br and I in the presence of a catalyst system to give intermediates of structure 10 (step b). An appropriate catalytic system for such a transformation consists for instance, but is not limited, of [Pd(PPPh₃)₂Cl₂]in the presence of LiOtBu in a solvent such as 1,4-dioxane at a temperature of 80° C. Heterogeneous catalytic hydrogenation of olefin 10 using a catalyst such as Pd(OH)₂ or Pd/C, preferably Pd/C in a solvent like THF, MeOH, EtOH, EtOAc or a mixture thereof, preferably in THF at around room temperature and under e.g., atmospheric pressure of hydrogen, affords intermediates of type 11 (step c). The BOC group of intermediate 11 is cleaved to give intermediates of type 1 using acidic conditions such as 4M HCl in dioxane in a solvent like MeOH, or, preferably, TFA in DCM at around room temperature (step d).

Ketones 7 and aryl halides 9 are commercially available or can be synthesized according to methods known to a person skilled in the art.

In one aspect, the present invention provides a process of manufacturing the compounds of formula (I) as described herein, comprising:
(a) reacting a first amine 4a,5,6,7,8,8a-hexahydro-4H-pyrido[4,3-b][1,4]oxazin-3-one (2),

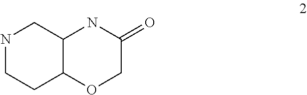 [SMILES: O=C1COC2CCNCC2N1]

with a second amine 1, wherein R², R³, R⁴, L and PG are as defined herein

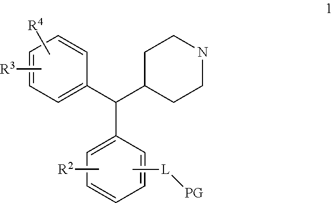

in the presence of a base and a urea forming reagent,
to form a compound of formula 3, wherein R², R³, R⁴, L and PG are as defined herein

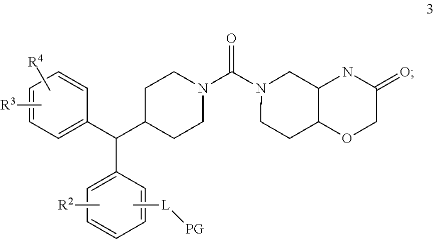

or
(b) reacting a first amine 4a,5,6,7,8,8a-hexahydro-4H-pyrido[4,3-b][1,4]oxazin-3-one (2),

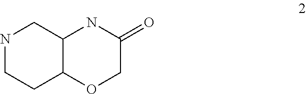 [SMILES: O=C1COC2CCNCC2N1]

with a second amine 1a, wherein R¹, R², R³, R⁴, and L are as defined herein

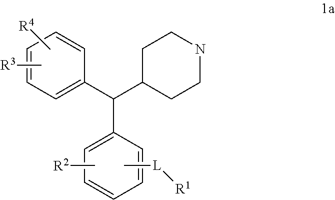

in the presence of a base and a urea forming reagent,
to form said compound of formula (I).

In one embodiment, there is provided a process according to the invention, wherein said base is sodium bicarbonate.

In one embodiment, there is provided a process according to the invention, wherein said urea forming reagent is selected from bis(trichloromethyl) carbonate, phosgene, trichloromethyl chloroformate, (4-nitrophenyl)carbonate and 1,1′-carbonyldiimidazole, preferably wherein said urea forming reagent is bis(trichloromethyl) carbonate.

In one embodiment, there is provided a process according to the invention, wherein said protective group “PG” is a Cbz or a Boc protective group.

In one aspect, the present invention provides a compound of formula (I) as described herein, when manufactured according to any one of the processes described herein.

### MAGL Inhibitory Activity

Compounds were profiled for MAGL inhibitory activity by determining the enzymatic activity by following the hydrolysis of the natural substrate 2-arachidonoylglycerol (2-AG) resulting in arachidonic acid, which can be followed by mass spectrometry. This assay is hereinafter abbreviated “2-AG assay”.

The 2-AG assay was carried out in 384 well assay plates (PP, Greiner Cat#784201) in a total volume of 20 μL. Compound dilutions were made in 100% DMSO (VWR Chemicals 23500.297) in a polypropylene plate in 3-fold dilution steps to give a final concentration range in the assay from 12.5 μM to 0.8 pM. 0.254 compound dilutions (100% DMSO) were added to 9 μL MAGL in assay buffer (50 mM TRIS (GIBCO, 15567-027), 1 mM EDTA (Fluka, 03690-100 mL), 0.01% (v/v) Tween. After shaking, the plate was incubated for 15 min at RT. To start the reaction, 10 μL 2-arachidonoylglycerol in assay buffer was added. The final concentrations in the assay was 50 pM MAGL and 8 μM 2-arachidonoylglyerol. After shaking and 30 min incubation at RT, the reaction was quenched by the addition of 404 of ACN containing 4 μM of d8-arachidonic acid. The amount of arachidonic acid was traced by an online SPE system (Agilent Rapidfire) coupled to a triple quadrupole mass spectrometer (Agilent 6460). A C18 SPE cartridge (G9205A) was used in an ACN/water liquid setup. The mass spectrometer was operated in negative electrospray mode following the mass transitions 303.1→259.1 for arachidonic acid and 311.1→267.0 for d8-arachidonic acid. The activity of the compounds was calculated based on the ratio of intensities [arachidonic acid/d8-arachidonic acid].

TABLE 1

| | IC₅₀ MAGL |
|---|---|
| Example | [nM] |
| 1 | 1.0 |
| 2 | 1.2 |
| 3 | 2.1 |
| 4 | 0.3 |
| 5 | 0.2 |
| 6 | 5.3 |
| 7 | 48.5 |
| 8 | 1.5 |
| 9 | 2.6 |
| 10 | 7.8 |
| 11 | 2925.6 |
| 12 | 0.5 |
| 13 | 1.6 |
| 14 | 1.3 |
| 15 | 3.2 |
| 16 | 18.1 |
| 17 | 0.7 |
| 18 | 7.2 |
| 19 | 2.0 |
| 20 | 11.4 |
| 21 | 0.3 |
| 22 | 0.4 |
| 23 | 5.8 |
| 24 | 5.5 |
| 25 | 13.0 |

In one aspect, the present invention provides compounds of formula (I) and their pharmaceutically acceptable salts or esters as described herein, wherein said compounds of formula (I) and their pharmaceutically acceptable salts or esters have IC₅₀'s for MAGL inhibition below 25 μM, preferably below 10 μM, more preferably below 5 μM as measured in the MAGL assay described herein.

In one embodiment, compounds of formula (I) and their pharmaceutically acceptable salts or esters as described herein have IC₅₀ (MAGL inhibition) values between 0.000001 μM and 25 μM, particular compounds have IC₅₀ values between 0.000005 μM and 10 μM, further particular compounds have IC₅₀ values between 0.00005 μM and 5 μM, as measured in the MAGL assay described herein.

Using the Compounds of the Invention

The compounds of formula (I) are fluorescent imaging probes with high affinity for MAGL. They may thus be used as high resolution tools to investigate localization, e.g. expression levels and protein distribution in health and disease, structure, dynamics and function of MAGL in living cells. They may also be applied e.g. in flow cytometry fluorescence-activated cell sorting (FACS) experiments or cellular trafficking studies using confocal live cell imaging.

In one aspect, the present invention provides a compound of formula (I) described herein, for use in monoacylglycerol lipase (MAGL) occupancy studies.

In a further aspect, the present invention provides a compound of formula (I) described herein, for use in diagnostic imaging of monoacylglycerol lipase (MAGL) in a mammal.

In a further aspect, the present invention provides a compound of formula (I) described herein, for use in generating monoacylglycerol lipase (MAGL) equilibrium and kinetic binding data.

In a further aspect, the present invention provides using a compound of formula (I) described herein in monoacylglycerol lipase (MAGL) occupancy studies.

In a further aspect, the present invention provides using a compound of formula (I) described herein in diagnostic imaging of monoacylglycerol lipase (MAGL) in a mammal.

In a further aspect, the present invention provides using a compound of formula (I) described herein for generating monoacylglycerol lipase (MAGL) equilibrium and kinetic binding data.

In a further aspect, the present invention provides a method of studying monoacylglycerol lipase (MAGL) occupancy, comprising contacting MAGL with a compound of formula (I) described herein.

In a further aspect, the present invention provides a method of diagnostic imaging of monoacylglycerol lipase (MAGL) in a mammal, comprising contacting MAGL with a compound of formula (I) described herein.

In a further aspect, the present invention provides a method of generating monoacylglycerol lipase (MAGL) equilibrium and kinetic binding data, comprising contacting MAGL with a compound of formula (I) described herein.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

### Example 1

### 3-(2-(2-(2-((7-Nitrobenzo[c][1,2,5]oxadiazol-4-yl)amino)ethoxy)ethoxy)ethoxy)-N-(2-(3-((1-((4aR,8aS)-3-oxooctahydro-2H-pyrido[4,3-b][1,4]oxazine-6-carbonyl)piperidin-4-yl)(phenyl)methyl)phenoxy)ethyl)propanamide

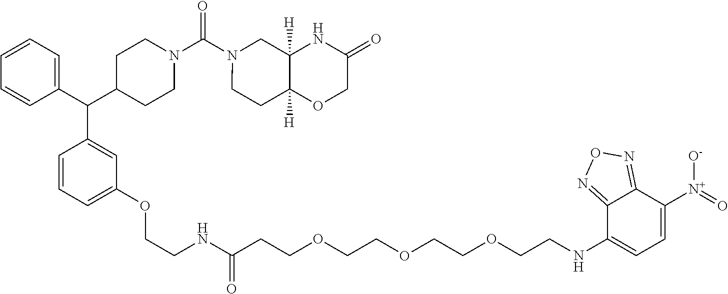 [SMILES: O=C(CCOCCOCCOCCNc1ccc([N+](=O)[O-])c2nonc12)NCCOc1cccc(C(c2ccccc2)C2CCN(C(=O)N3CC[C@@H]4OCC(=O)N[C@@H]4C3)CC2)c1]

### Step a) benzyl-(Z)-4-(Phenyl(2-tosylhydrazineylidene)methyl)piperidine-1-carboxylate

A solution of 4-methylbenzenesulfonhydrazide (1.54 g, 8.29 mmol; CAS RN 1576-35-8) and tert-butyl 4-benzoylpiperidine-1-carboxylate (2.0 g, 6.91 mmol; CAS RN 922504-27-6) in 1,4-dioxane (150 mL) was stirred at 100° C. for 16 hours. EtOAc and H₂O water were added. The layers were separated and the organic layer dried over Na₂SO₄. The solvent was removed under reduced pressure and the crude was purified by flash chromatography (ISCO silica gel, 25 g cartridge, Hept AcOEt 0-30) to provide the title compound (3.55 g, quant.) as light brown oil. LC-HRMS (ESI): 492.1945 ([M+H]⁺).

### Step b) Benzyl 4-((3-(2-((tert-butoxycarbonyl)amino)ethoxy)phenyl)(phenyl)methylene)piperidine-1-carboxylate

A mixture of benzyl (Z)-4-(phenyl(2-tosylhydrazineylidene)methyl)piperidine-1-carboxylate (1 g, 2.03 mmol), bis(triphenylphosphine)palladium(II) chloride (143 mg, 203 μmol), lithium tert-butoxide (246 mg, 3.05 mmol) and N-Boc-2-(3-bromophenoxy)ethylamine (772 mg, 2.44 mmol; CAS RN 1098107-26-6) in 1,4-dioxane (3 mL) was stirred at 80° C. for 14 hours. EtOAc and H₂O water were added. The layers were separated and the organic layer dried over Na₂SO₄. The solvent was removed under reduced pressure and the crude was purified by flash chromatography (ISCO silica gel, 80 g cartridge, Hept AcOEt 0-30) to provide the title compound (280 mg, 22%) as light yellow foam. LC-HRMS (ESI): 443.2309 ([M−Boc+H]⁺).

### Step c) tert-Butyl (2-(3-(phenyl(piperidin-4-yl)methyl)phenoxy)ethyl)carbamate

Pd+Pt/C (2.5%+2.5%, 7.01 g) was added to a solution of benzyl 4-4342-((tert-butoxycarbonyl)amino)ethoxy)phenyl)(phenyl)methylene)piperidine-1-carboxylate (4.8 g, 8.85 mmol) in methanol (240 mL) and AcOH (4.8 mL) in an autoclave. The reaction vessel was purged three times with hydrogen gas. The mixture was stirred for 18 hours at 50° C. under 50 bar hydrogen gas pressure. The catalyst was filtered off and washed with methanol. The filtrate was concentrated in vacuo to provide the title compound (3.386 g, 93%) as colorless solid. LC-HRMS (ESI): 411.2644 ([M+H]⁺).

### Step d) (+)-cis-4a,5,6,7,8,8a-Hexahydro-4H-pyrido[4,3-b][1,4]oxazin-3-one

The enantiomers of rac-(4aR,8aS)-hexahydro-2H-pyrido[4,3-b][1,4]oxazin-3(4H)-one dihydrochloride (500 mg, 2.18 mmol, ChemBridge Corporation) were separated by preparative chiral HPLC (ReprosilChiral NR column) using an isocratic mixture of EtOH (containing 0.05% of NH₄OAc):n-heptane (30:70).

First eluting enantiomer: (+)-cis-4a,5,6,7,8,8a-Hexahydro-4H-pyrido[4,3-b][1,4]oxazin-3-one. Yellow solid (0.150 g; 44.0%). MS (ESI): m/z=157.1 [M+H]+.

Second eluting enantiomer: (−)-cis-4a,5,6,7,8,8a-Hexahydro-4H-pyrido[4,3-b][1,4]oxazin-3-one. Yellow solid (0.152 g; 44.6%). MS (ESI): m/z=157.1 [M+H]+.

### Step e) tert-Butyl (2-(3-((1-(4aR,8aS)-3-oxooctahydro-2H-pyrido[4,3-b][1,4]oxazine-6-carbonyl)piperidin-4-yl)(phenyl)methyl)phenoxy)ethyl)carbamate

A mixture of (+)-cis-4a,5,6,7,8,8a-hexahydro-4H-pyrido[4,3-b][1,4]oxazin-3-one (295 mg, 1.1 mmol) and sodium bicarbonate (368 mg, 4.38 mmol) in DCM (13 mL) was cooled to 0° C. Triphosgene (228 mg, 767 μmol) was added and the mixture was stirred at ambient temperature for 14 hours and cooled to 0° C. again. tert-Butyl (2-(3-(phenyl(piperidin-4-yl)methyl)phenoxy)ethyl)carbamate (450 mg, 1.1 mmol) and DIPEA (567 mg, 766 μL, 4.38 mmol) were added. The suspension was stirred at ambient temperature for 2 hours. EtOAc and H₂O water were added. The layers were separated and the organic layer dried over Na₂SO₄. The solvent was removed under reduced pressure and the crude was purified by preparative HPLC to provide the title compound (392 mg, 60%) as off-white foam. MS (ESI): m/z=493.4 ([M−Boc+H]⁺).

### Step f) (4aR,8aS)-6-(4-(3-(2-Aminoethoxy)phenyl)(phenyl)methyl)piperidine-1-carbonyl)hexahydro-2H-pyrido[4,3-b][1,4]oxazin-3(4H)-one

TFA (274 mg, 185 μL, 2.4 mmol) was added to a solution of tert-butyl (2-(3-((1-((4aR,8aS)-3-oxooctahydro-2H-pyrido[4,3-b][1,4]oxazine-6-carbonyl)piperidin-4-yl)(phenyl)methyl)phenoxy)ethyl)carbamate (178 mg, 300 μmol) in DCM (2.49 mL). The reaction mixture was stirred at ambient temperature for 3 hours. Ethyl acetate and aqueous KHCO₃ solution were added. The layers were separated. The aqueous layer was extracted with ethyl acetate. The combined organic phases were dried over sodium sulfate and brought to dryness under reduced pressure to obtain the title compound (178 mg, quant.) as light yellow foam which was used in the next step without further purification. LC-HRMS (ESI): 493.2813 ([M+H]⁺).

### Step g) tert-Butyl (12-oxo-15-(3-(1-(4aR,8aS)-3-oxooctahydro-2H-pyrido[4,3-b][1,4]oxazine-6-carbonyl)piperidin-4-yl)(phenyl)methyl)phenoxy)-3,6,9-trioxa-13-azapentadecyl)carbamate

DIPEA (52.5 mg, 70.9 μL, 406 μmol) was added to a mixture of (4aR,8aS)-6-(4-((3-(2-aminoethoxy)phenyl)(phenyl)methyl)piperidine-1-carbonyl)hexahydro-2H-pyrido[4,3-b][1,4]oxazin-3(4H)-one (50 mg, 101 μmol), 3-[2-[2-[2-(tert-butoxycarbonylamino)ethoxy]ethoxy]ethoxy]propanoic acid (33 mg, 101 μmol; CAS RN 1347750-75-7) and HATU (43 mg, 112 μmol) in DMF (338 μL). The mixture was stirred at ambient temperature for 18 h. Ethyl acetate and aqueous KHCO₃ solution were added. The layers were separated. The organic layer was washed with water and dried over sodium sulfate. Removal of the solvent provided crude product which was purified by flash chromatography (ISCO silica gel, 12 g cartridge, Hept EtOAc 0-70% and DCM MeOH 0-10%) to obtain the title compound (36 mg, 48%) as colorless solid. LC-HRMS (ESI): 796.4494 ([M+H]⁺).

### Step h) 3-(2-(2-(2-Aminoethoxy)ethoxy)ethoxy)-N-(2-(3-((1-(4aR,8aS)-3-oxooctahydro-2H-pyrido[4,3-b][1,4]oxazine-6-carbonyl)piperidin-4-yl)(phenyl)methyl)phenoxy)ethyl)propanamide

TFA (51 mg, 34.4 μL, 446 μmol was added to a solution of tert-butyl (12-oxo-15-(3-((1-((4aR,8aS)-3-oxooctahydro-2H-pyrido[4,3-b][1,4]oxazine-6-carbonyl)piperidin-4-yl)(phenyl)methyl)phenoxy)-3,6,9-trioxa-13-azapentadecyl)carbamate (74 mg, 56 μmol in CH₂Cl₂ (21 μL). The reaction mixture was stirred at ambient temperature for 3 hours. Ethyl acetate and aqueous KHCO₃ solution were added. The layers were separated. The aqueous layer was extracted with ethyl acetate. The combined organic phases were dried over sodium sulfate and brought to dryness under reduced pressure to obtain the title compound (36 mg, 48%) as light yellow foam. LC-HRMS (ESI): 696.3963 ([M+H]⁺).

### Step i) 3-(2-(2-(24(7-Nitrobenzo[c][1,2,5]oxadiazol-4-yl)amino)ethoxy)ethoxy)ethoxy)-N-(2-(3-((14(4aR,8aS)-3-oxooctahydro-2H-pyrido[4,3-b][1,4]oxazine-6-carbonyl)piperidin-4-yl)(phenyl)methyl)phenoxy)ethyl)propanamide

4-Chloro-7-nitrobenz-2-oxa-1,3-diazole (2.8 mg, 14 μmol; CAS RN 10199-89-0) and N,N-diisopropylethylamine (1.9 mg, 2.5 μL, 14 μmol) were added to a solution of 3-(2-(2-(2-aminoethoxy)ethoxy)ethoxy)-N-(2-(3-((1-((4aR,8aS)-3-oxooctahydro-2H-pyrido[4,3-b][1,4]oxazine-6-carbonyl)piperidin-4-yl)(phenyl)methyl)phenoxy)ethyl)propanamide (10 mg, 14.4 μmol in MeOH (240 μL). The reaction mixture was stirred for 14 h at ambient temperature and poured onto ethyl acetate/water. The layers were separated. The the organic layer was dried over sodium sulfate and evaporated to dryness. The crude material was purified by flash chromatography (ISCO silica gel, 25 g cartridge, DCM MeOH 0-40%) to obtain the title compound (5 mg, 34%) as orange solid. LC-HRMS (ESI): 859.3985 ([M+H]⁺).

### Example 2

### 3-(2-((7-Nitrobenzo[c][1,2,5]oxadiazol-4-yl)amino)ethoxy)-N-(2-(3-((1-04aR,8aS)-3-oxooctahydro-2H-pyrido[4,3-b][1,4]oxazine-6-carbonyl)piperidin-4-yl)(phenyl)methyl)phenoxy)ethyl)propanamide

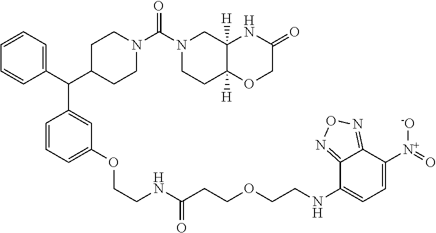

### Step a) tert-Butyl (2-(3-oxo-3-((2-((4-((1-(4aR,8aS)-3-oxooctahydro-2H-pyrido[4,3-b][1,4]oxazine-6-carbonyl)piperidin-4-yl)(phenyl)methyl)phenoxy)ethyl)amino)propoxy)ethyl)carbamate

In analogy to the procedure described in example 1 g), (4aR,8aS)-6-(4-((3-(2-aminoethoxy)phenyl)(phenyl)methyl)piperidine-1-carbonyl)hexahydro-2H-pyrido[4,3-b][1,4]oxazin-3(4H)-one (example 1 f) was condensed with 3-[2-(tert-butoxycarbonylamino)ethoxy]propanoic acid (CAS RN 1260092-44-1) to obtain the title compound as colorless oil.

### Step b) 3-(2-Aminoethoxy)-N-(2-(3-(1-(4aR,8aS)-3-oxooctahydro-2H-pyrido[4,3-b][1,4]oxazine-6-carbonyl)piperidin-4-yl)(phenyl)methyl)phenoxy)ethyl)propanamide

In analogy to the procedure described in example 1 h), tert-butyl (2-(3-oxo-3-((2-(3-((1-((4aR,8aS)-3-oxooctahydro-2H-pyrido[4,3-b][1,4]oxazine-6-carbonyl)piperidin-4-yl)(phenyl)methyl)phenoxy)ethyl)amino)propoxy)ethyl)carbamate was deprotected with TFA to obtain the title compound as off-white solid. LC-HRMS (ESI): 608.3437 ([M+H]⁺).

### Step c) 3-(2-((7-Nitrobenzo[c][1,2,5]oxadiazol-4-yl)amino)ethoxy)-N-(2-(3-((1-((4aR,8aS)-3-oxooctahydro-2H-pyrido[4,3-b][1,4]oxazine-6-carbonyl)piperidin-4-yl)(phenyl)methyl)phenoxy)ethyl)propanamide

In analogy to the procedure described in example 1 i), 3-(2-aminoethoxy)-N-(2-(3-((1-((4aR,8aS)-3-oxooctahydro-2H-pyrido[4,3-b][1,4]oxazine-6-carbonyl)piperidin-4-yl)(phenyl)methyl)phenoxy)ethyl)propanamide was reacted with 4-chloro-7-nitrobenz-2-oxa-1,3-diazole to obtain the title compound as orange solid. LC-HRMS (ESI): 770.3461 ([M+H]⁺).

### Example 3

### 3-(2-(2-((7-Nitrobenzo[c][1,2,5]oxadiazol-4-yl)amino)ethoxy)ethoxy)-N-(2-(3-((1-((4aR,8aS)-3-oxooctahydro-2H-pyrido[4,3-b][1,4]oxazine-6-carbonyl)piperidin-4-yl)(phenyl)methyl)phenoxy)ethyl)propanamide

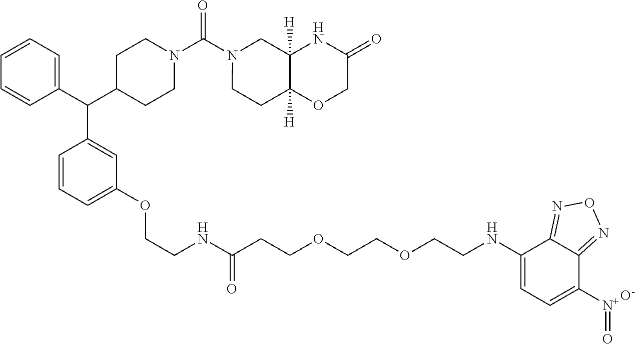

### Step a) tert-Butyl (2-(2-(3-oxo-3-((2-(3-(1-(4aR,8aS)-3-oxooctahydro-2H-pyrido[4,3-b][1,4]oxazine-6-carbonyl)piperidin-4-yl)(phenyl)methyl)phenoxy)ethyl)amino)propoxy)ethoxy)ethyl)carbamate

In analogy to the procedure described in example 1 g), (4aR,8aS)-6-(4-((3-(2-aminoethoxy)phenyl)(phenyl)methyl)piperidine-1-carbonyl)hexahydro-2H-pyrido[4,3-b][1,4]oxazin-3(4H)-one (example 1 f) was condensed with 3-[2-[2-(tert-butoxycarbonylamino)ethoxy]ethoxy]propanoic acid (CAS RN 1365655-91-9) to obtain the title compound as colorless oil. LC-HRMS (ESI): 774.4065 ([M+Na]⁺).

### Step b) 3-(2-(2-Aminoethoxy)ethoxy)-N-(2-(3-(1-(4aR,8aS)-3-oxooctahydro-2H-pyrido[4,3-b][1,4]oxazine-6-carbonyl)piperidin-4-yl)(phenyl)methyl)phenoxy)ethyl)propanamide

In analogy to the procedure described in example 1 h), tert-butyl (2-(2-(3-oxo-3-((2-(3-((1-((4aR,8aS)-3-oxooctahydro-2H-pyrido[4,3-b][1,4]oxazine-6-carbonyl)piperidin-4-yl)(phenyl)methyl)phenoxy)ethyl)amino)propoxy)ethoxy)ethyl)carbamate was deprotected with TFA to obtain the title compound as off-white solid. LC-HRMS (ESI): 652.3702 ([M+H]⁺).

### Step c) 3-(2-(2-((7-Nitrobenzo[c][1,2,5]oxadiazol-4-yl)amino)ethoxy)ethoxy)-N-(2-(3-((1-((4aR,8aS)-3-oxooctahydro-2H-pyrido[4,3-b][1,4]oxazine-6-carbonyl)piperidin-4-yl)(phenyl)methyl)phenoxy)ethyl)propanamide

In analogy to the procedure described in example 1 i), 3-(2-(2-aminoethoxy)ethoxy)-N-(2-(3-((1-((4aR,8aS)-3-oxooctahydro-2H-pyrido[4,3-b][1,4]oxazine-6-carbonyl)piperidin-4-yl)(phenyl)methyl)phenoxy)ethyl)propanamide was reacted with 4-chloro-7-nitrobenz-2-oxa-1,3-diazole to obtain the title compound as orange solid. LC-HRMS (ESI): 814.3638 ([M+H]⁺).

### Example 4

### tert-Butyl N-[2-[3-[(R or S)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate

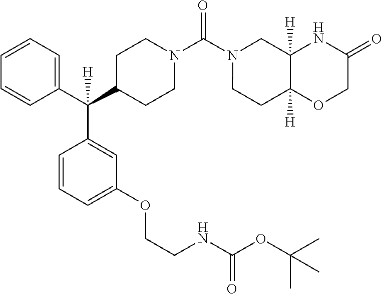 [SMILES: CC(C)(C)OC(=O)NCCOc1cccc([C@@H](c2ccccc2)C2CCN(C(=O)N3CC[C@@H]4OCC(=O)N[C@@H]4C3)CC2)c1]

### Step a) tert-Butyl N-[2-[3-[(R or S)-phenyl(4-piperidyl)methyl]phenoxy]ethyl]carbamate

The enantiomers of rac-tert-butyl (2-(3-(phenyl(piperidin-4-yl)methyl)phenoxy)ethyl)carbamate (example 1 c) were separated by preparative chiral HPLC.

First eluting enantiomer: (−)-tert-Butyl N-[2-[3-[(R or S)-phenyl(4-piperidyl)methyl]phenoxy]ethyl]carbamate. White foam. LC-HRMS (ESI): 411.2648 ([M+H]⁺).

Second eluting enantiomer: (+)-tert-Butyl N-[2-[3-[(S or R)-phenyl(4-piperidyl)methyl]phenoxy]ethyl]carbamate. White foam. LC-HRMS (ESI): 411.2649 ([M+H]⁺).

### Step b) tert-Butyl N-[2-[3-[(R or S)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate

In analogy to the procedure described in example 1 e), (+)-cis-4a,5,6,7,8,8a-hexahydro-4H-pyrido[4,3-b][1,4]oxazin-3-one (example 1 d) was reacted with tert-butyl N-[2-[3-[(R or 5)-phenyl(4-piperidyl)methyl]phenoxy]ethyl]carbamate to obtain the title compound as colorless oil. LC-HRMS (ESI): 593.3335 ([M+H]⁺).

### Example 5

### tert-Butyl N-[2-[3-[(S or R)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate

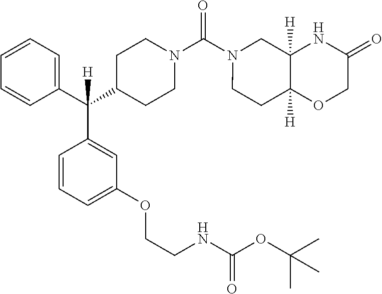 [SMILES: CC(C)(C)OC(=O)NCCOc1cccc([C@H](c2ccccc2)C2CCN(C(=O)N3CC[C@@H]4OCC(=O)N[C@@H]4C3)CC2)c1]

### Step a) tert-Butyl N-[2-[3-[(S or R)-phenyl(4-piperidyl)methyl]phenoxy]ethyl]carbamate

The enantiomers of rac-tert-butyl (2-(3-(phenyl(piperidin-4-yl)methyl)phenoxy)ethyl)carbamate (example 1 c) were separated by preparative chiral HPLC.

First eluting enantiomer: (−)-tert-Butyl N-[2-[3-[(R or S)-phenyl(4-piperidyl)methyl]phenoxy]ethyl]carbamate. White foam. LC-HRMS (ESI): 411.2648 ([M+H]⁺).

Second eluting enantiomer: (+)-tert-Butyl N-[2-[3-[(S or R)-phenyl(4-piperidyl)methyl]phenoxy]ethyl]carbamate. White foam. LC-HRMS (ESI): 411.2649 ([M+H]⁺).

### Step b) tert-Butyl N-[2-[3-[(S or R)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate

In analogy to the procedure described in example 1 e), (+)-cis-4a,5,6,7,8,8a-hexahydro-4H-pyrido[4,3-b][1,4]oxazin-3-one (example 1 d) was reacted with tert-butyl N-[2-[3-[(S or R)-phenyl(4-piperidyl)methyl]phenoxy]ethyl]carbamate to obtain the title compound as off-white foam. LC-HRMS (ESI): 593.3336 ([M+H]⁺).

### Example 6

### (4aR,8aS)-6-[4-[(S or R)-[3-(2-Aminoethoxy)phenyl]-phenyl-methyl]piperidine-1-carbonyl]-4,4a,5,7,8,8a-hexahydropyrido[4,3-b][1,4]oxazin-3-one

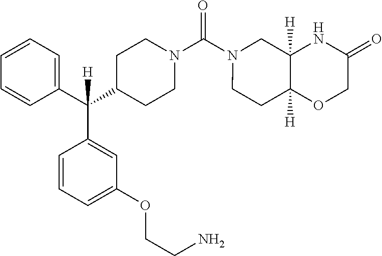 [SMILES: NCCOc1cccc([C@H](c2ccccc2)C2CCN(C(=O)N3CC[C@@H]4OCC(=O)N[C@@H]4C3)CC2)c1]

In analogy to the procedure described in example 1 f), tert-butyl N-[2-[3-[(S or R)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate (example 5 b) was deprotected with TFA to obtain the title compound as yellow oil. LC-HRMS (ESI): 493.2815 ([M+H]⁺).

### Example 7

### (4aR,8aS)-6-[4-[(R or S)-[3-(2-Aminoethoxy)phenyl]-phenyl-methyl]piperidine-1-carbonyl]-4,4a,5,7,8,8a-hexahydropyrido[4,3-b][1,4]oxazin-3-one

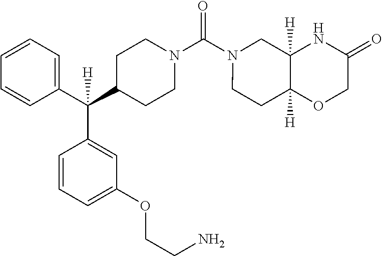 [SMILES: NCCOc1cccc([C@@H](c2ccccc2)C2CCN(C(=O)N3CC[C@@H]4OCC(=O)N[C@@H]4C3)CC2)c1]

In analogy to the procedure described in example 1f), tert-butyl N-[2-[3-[(R or S)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate (example 4 b) was deprotected with TFA to obtain the title compound as yellow oil. LC-HRMS (ESI): 493.2815 ([M+H]⁺).

### Example 8

### tert-Butyl N-[2-[2-[3-[2-[3-[(S or R)-11-1(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethoxy]ethyl]carbamate

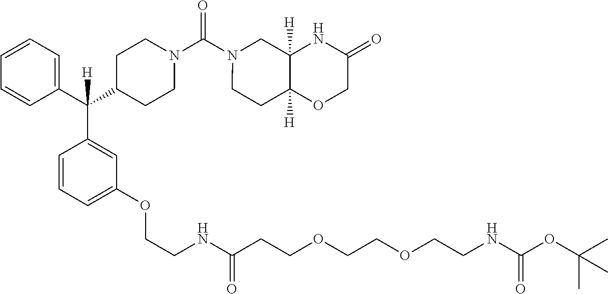 [SMILES: CC(C)(C)OC(=O)NCCOCCOCCC(=O)NCCOc1cccc([C@H](c2ccccc2)C2CCN(C(=O)N3CC[C@@H]4OCC(=O)N[C@@H]4C3)CC2)c1]

In analogy to the procedure described in example 1 g), (4aR,8aS)-6-[4-[(S or R)-[3-(2-aminoethoxy)phenyl]-phenyl-methyl]piperidine-1-carbonyl]-4,4a,5,7,8,8a-hexahydropyrido[4,3-b][1,4]oxazin-3-one (example 6) was condensed with 3-[2-[2-(tert-butoxycarbonylamino)ethoxy]ethoxy]propanoic acid (CAS RN 1365655-91-9) to obtain the title compound as white powder. LC-HRMS (ESI): 752.4237 ([M+H]⁺).

### Example 9

### tert-Butyl N-[2-[2-[3-[2-[3-[(R or S)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethoxy]ethyl]carbamate

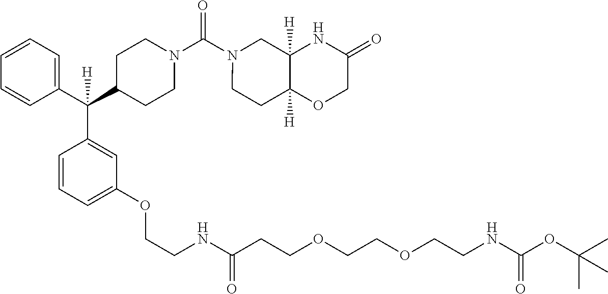 [SMILES: CC(C)(C)OC(=O)NCCOCCOCCC(=O)NCCOc1cccc([C@@H](c2ccccc2)C2CCN(C(=O)N3CC[C@@H]4OCC(=O)N[C@@H]4C3)CC2)c1]

In analogy to the procedure described in example 1 g), (4aR,8aS)-6-[4-[(R or S)-[3-(2-aminoethoxy)phenyl]-phenyl-methyl]piperidine-1-carbonyl]-4,4a,5,7,8,8a-hexahydropyrido[4,3-b][1,4]oxazin-3-one (example 7) was condensed with 34242-(tert-butoxycarbonylamino)ethoxy]ethoxy]propanoic acid (CAS RN 1365655-91-9) to obtain the title compound as white powder. LC-HRMS (ESI): 752.4236 ([M+H]⁺).

### Example 10

### N-[2-[3-[(R or S)-[1-[(4aR,8aS)-3-Oxo-4,4a,5,7,8,8a-hexahydropyrido[4,3-b][1,4]oxazine-6-carbonyl]-4-piperidyl]-phenyl-methyl]phenoxy]ethyl]-3-[2-(2-aminoethoxy)ethoxy]propanamide

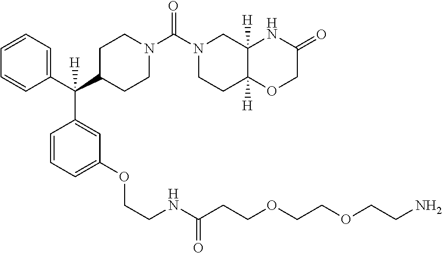 [SMILES: NCCOCCOCCC(=O)NCCOc1cccc([C@@H](c2ccccc2)C2CCN(C(=O)N3CC[C@@H]4OCC(=O)N[C@@H]4C3)CC2)c1]

In analogy to the procedure described in example 1 h), tert-butyl N-[2-[2-[3-[2-[3-[(R or S)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl amino]-3-oxo-propoxy]ethoxy]ethyl]carbamate (example 9) was deprotected with TFA to obtain the title compound as light yellow oil. LC-HRMS (ESI): 652.3703 ([M+H]⁺).

### Example 11

### N-[2-[3-[(S or R)-[1-[(4aR,8aS)-3-Oxo-4,4a,5,7,8,8a-hexahydropyrido[4,3-b][1,4]oxazine-6-carbonyl]-4-piperidyl]-phenyl-methyl]phenoxy]ethyl]-3-[2-(2-aminoethoxy)ethoxy]propanamide

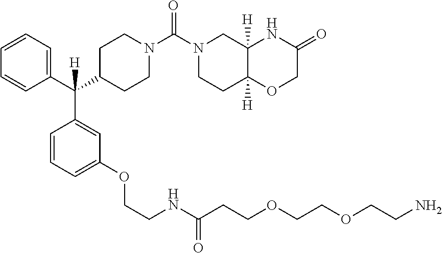 [SMILES: NCCOCCOCCC(=O)NCCOc1cccc([C@H](c2ccccc2)C2CCN(C(=O)N3CC[C@@H]4OCC(=O)N[C@@H]4C3)CC2)c1]

In analogy to the procedure described in example 1 h), tert-butyl N-[2-[2-[3-[2-[3-[(S or R)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl amino]-3-oxo-propoxy]ethoxy]ethyl]carbamate (example 8) was deprotected with TFA to obtain the title compound as light yellow oil. LC-HRMS (ESI): 652.3705 ([M+H]⁺).

### Example 12

### N-[2-[3-[(S or R)-[1-[(4aR,8aS)-3-Oxo-4,4a,5,7,8,8a-hexahydropyrido[4,3-b][1,4]oxazine-6-carbonyl]-4-piperidyl]-phenyl-methyl]phenoxy]ethyl]-3-[2-[2-[(4-nitro-2,1,3-benzoxadiazol-7-yl)amino]ethoxy]ethoxy]propanamide

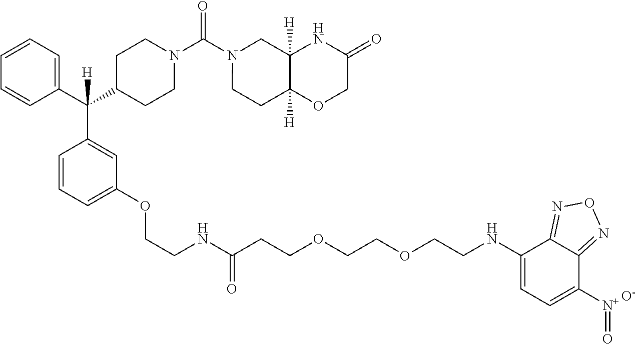

In analogy to the procedure described in example 1 i), N-[2-[3-[(S or R)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-(2-aminoethoxy)ethoxy]propanamide (example 11) was reacted with 4-chloro-7-nitrobenz-2-oxa-1,3-diazole to obtain the title compound as yellow solid. LC-HRMS (ESI): 813.3573 ([M−H]).

### Example 13

### N-[2-[3-[(R or S)-[1-[(4aR,8aS)-3-Oxo-4,4a,5,7,8,8a-hexahydropyrido[4,3-b][1,4]oxazine-6-carbonyl]-4-piperidyl]phenyl-methyl]phenoxy]ethyl]-3-[2-[2-[(4-nitro-2,1,3-benzoxadiazol-7-yl)amino]ethoxy]ethoxy]propanamide

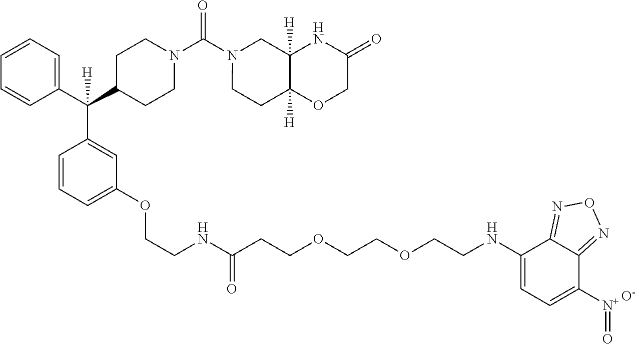

In analogy to the procedure described in example 1 i), N-[2-[3-[(R or S)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-(2-aminoethoxy)ethoxy]propanamide (example 10) was reacted with 4-chloro-7-nitrobenz-2-oxa-1,3-diazole to obtain the title compound as yellow solid. LC-HRMS (ESI): 813.3571 ([M−H]).

### Example 14

### tert-Butyl N-[2-[3-[2-[3-[(S or R)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethyl]carbamate

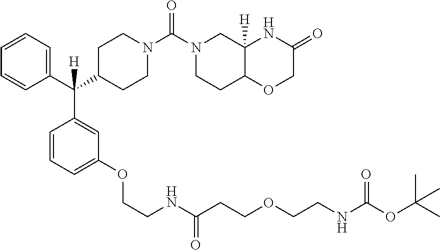 [SMILES: CC(C)(C)OC(=O)NCCOCCC(=O)NCCOc1cccc([C@H](c2ccccc2)C2CCN(C(=O)N3CCC4OCC(=O)N[C@@H]4C3)CC2)c1]

In analogy to the procedure described in example 1 g), (4aR,8aS)-6-[4-[(S or R)-[3-(2-aminoethoxy)phenyl]-phenyl-methyl]piperidine-1-carbonyl]-4,4a,5,7,8,8a-hexahydropyrido[4,3-b][1,4]oxazin-3-one (example 6) was condensed with 3-[2-(tert-butoxycarbonylamino)ethoxy]propanoic acid (CAS RN 1260092-44-1) to obtain the title compound as colorless oil. LC-HRMS (ESI): 708.3957 ([M+H]⁺).

### Example 15

### tert-Butyl N-[2-[3-[2-[3-[(R or S)-[1-1(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethyl]carbamate

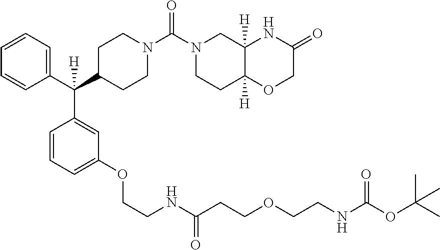 [SMILES: CC(C)(C)OC(=O)NCCOCCC(=O)NCCOc1cccc([C@@H](c2ccccc2)C2CCN(C(=O)N3CC[C@@H]4OCC(=O)N[C@@H]4C3)CC2)c1]

In analogy to the procedure described in example 1 g), (4aR,8aS)-6-[4-[(R or S)-[3-(2-aminoethoxy)phenyl]-phenyl-methyl]piperidine-1-carbonyl]-4,4a,5,7,8,8a-hexahydropyrido[4,3-b][1,4]oxazin-3-one (example 7) was condensed with 3-[2-(tert-butoxycarbonylamino)ethoxy]propanoic acid (CAS RN 1260092-44-1) to obtain the title compound as colorless oil. LC-HRMS (ESI): 708.3957 ([M+H]⁺).

### Example 16

### N-[2-[3-[(R or S)-[1-1(4aR,8aS)-3-Oxo-4,4a,5,7,8,8a-hexahydropyrido[4,3-b][1,4]oxazine-6-carbonyl]-4-piperidyl]phenyl-methyl]phenoxy]ethyl]-3-(2-aminoethoxy)propanamide

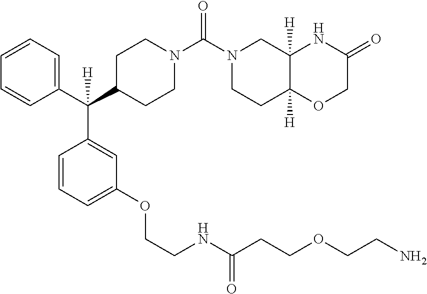 [SMILES: NCCOCCC(=O)NCCOc1cccc([C@@H](c2ccccc2)C2CCN(C(=O)N3CC[C@@H]4OCC(=O)N[C@@H]4C3)CC2)c1]

In analogy to the procedure described in example 1 h), tert-butyl N-[2-[3-[2-[3-[(R or S)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethyl]carbamate (example 15) was deprotected with TFA to obtain the title compound as light yellow oil. LC-HRMS (ESI): 608.3442 ([M+H]⁺).

### Example 17

### N-[2-[3-[(S or R)-11-1(4aR,8aS)-3-Oxo-4,4a,5,7,8,8a-hexahydropyrido[4,3-b][1,4]oxazine-6-carbonyl]-4-piperidyl]-phenyl-methyl]phenoxy]ethyl]-3-[2-[(4-nitro-2,1,3-benzoxadiazol-7-yl)amino]ethoxy]propanamide

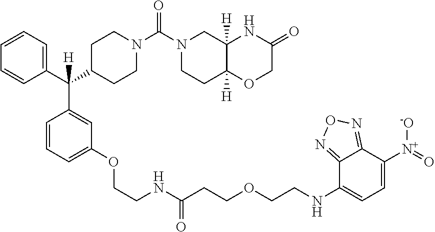

### Step a) N-[2-[3-[(S or R)-[1-[(4aR,8aS)-3-Oxo-4,4a,5,7,8,8a-hexahydropyrido[4,3-b][1,4]oxazine-6-carbonyl]-1-4-piperidyl]-phenyl-methyl]phenoxy]ethyl]-3-(2-aminoethoxy)propanamide

In analogy to the procedure described in example 1 h), tert-butyl N-[2-[3-[2-[3-[(S or R)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethyl]carbamate (example 14) was deprotected with TFA to obtain the title compound as light yellow oil. LC-HRMS (ESI): 608.3441 ([M+H]⁺).

### Step b) N-[2-[3-[(S or R)-[1-[(4aR,8aS)-3-Oxo-4,4a,5,7,8,8a-hexahydropyrido[4,3-b][1,4]oxazine-6-carbonyl]-4-piperidyl]-phenyl-methyl]phenoxy]ethyl]-3-(2-[(4-nitro-2,1,3-benzoxadiazol-7-yl)amino]ethoxy]propanamide

In analogy to the procedure described in example 1 i), N-[2-[3-[(R or S)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-(2-aminoethoxy)propanamide was reacted with 4-chloro-7-nitrobenz-2-oxa-1,3-diazole to obtain the title compound as yellow solid. LC-HRMS (ESI): 771.3464 ([M+H]⁺).

### Example 18

### N-[2-[3-[(R or S)-[1-1(4aR,8aS)-3-Oxo-4,4a,5,7,8,8a-hexahydropyrido[4,3-b][1,4]oxazine-6-carbonyl]-4-piperidyl]-phenyl-methyl]phenoxy]ethyl]-3-12-[(4-nitro-2,1,3-benzoxadiazol-7-yl)amino]ethoxy]propanamide

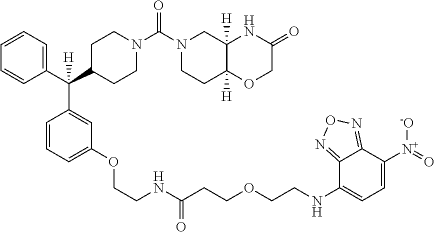

In analogy to the procedure described in example 1 i), N-[2-[3-[(R or S)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-(2-aminoethoxy)propanamide (example 16) was reacted with 4-chloro-7-nitrobenz-2-oxa-1,3-diazole to obtain the title compound as yellow solid. LC-HRMS (ESI): 771.3464 ([M+H]⁺).

### Example 19

### tert-Butyl N-[2-[2-[2-[3-[2-[3-[(S or R)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethoxy]ethoxy]ethyl]carbamate

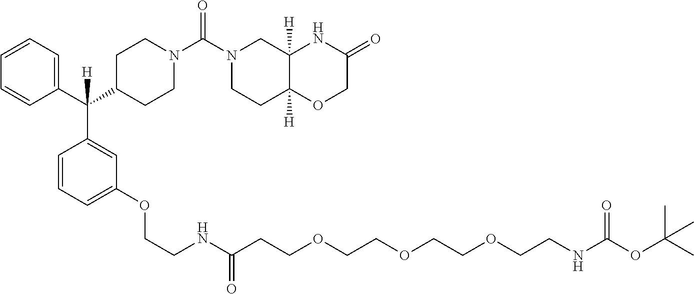 [SMILES: CC(C)(C)OC(=O)NCCOCCOCCOCCC(=O)NCCOc1cccc([C@H](c2ccccc2)C2CCN(C(=O)N3CC[C@@H]4OCC(=O)N[C@@H]4C3)CC2)c1]

In analogy to the procedure described in example 1 g), (4aR,8aS)-6-[4-[(S or R)-[3-(2-aminoethoxy)phenyl]-phenyl-methyl]piperidine-1-carbonyl]-4,4a,5,7,8,8a-hexahydropyrido[4,3-b][1,4]oxazin-3-one (example 7) was condensed with 3-[2-[2-[2-(tert-butoxycarbonylamino)ethoxy]ethoxy]ethoxy]propanoic acid (CAS RN 1347750-75-7) to obtain the title compound as brown oil. LC-HRMS (ESI): 796.4466 ([M+H]⁺).

### Example 20

### N-[2-[3-[(S or R)-[1-1(4aR,8aS)-3-Oxo-4,4a,5,7,8,8a-hexahydropyrido[4,3-b]11,4]oxazine-6-carbonyl]-4-piperidyfl-phenyl-methyl]phenoxy]ethyl]-3-[2-[2-(2-aminoethoxy)ethoxy]ethoxy]propanamide

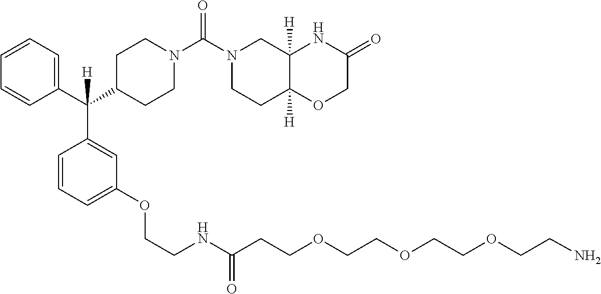 [SMILES: NCCOCCOCCOCCC(=O)NCCOc1cccc([C@H](c2ccccc2)C2CCN(C(=O)N3CC[C@@H]4OCC(=O)N[C@@H]4C3)CC2)c1]

In analogy to the procedure described in example 1 h), tert-butyl N-[2-[2-[2-[3-[2-[3-[(S or R)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethoxy]ethoxy]ethyl]carbamate (example 19) was deprotected with TFA to obtain the title compound as yellow oil. LC-HRMS (ESI): 696.3961 ([M+H]⁺).

### Example 21

### N-[2-[3-[2-[3-[(S or R)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethyl]-3-12,2-difluoro-12-(1H-pyrrol-2-yl)-3-aza-1-azonia-2-boranuidatricyclo[7.3.0.03,7]dodeca-1(12),4,6,8,10-pentaen-4-yl]propanamide

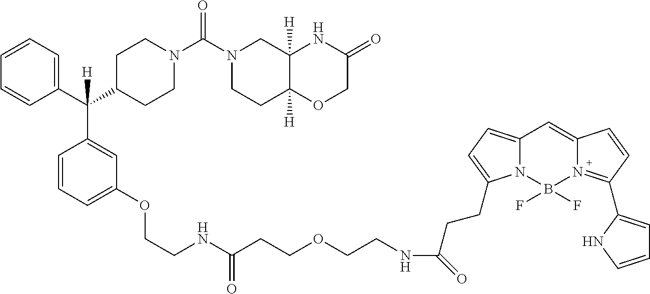

To a stirring solution of 3-(2-aminoethoxy)-N-(2-(3-((S)-(1-((4aR,8aS)-3-oxooctahydro-2H-pyrido[4,3-b][1,4]oxazine-6-carbonyl)piperidin-4-yl)(phenyl)methyl)phenoxy)ethyl)propanamide (example 17a, 3.4 mg, 5.6 μmol) in DMF (0.5 mL) at room temperature, 7-(3-((2,5-dioxopyrrolidin-1-yl)oxy)-3-oxopropyl)-5,5-difluoro-3-(1H-pyrrol-2-yl)-5H-514-dipyrrolo[1,2-c:2′,1′-f][1,3,2]diazaborinin-4-ium (2 mg, 4.7 μmol; CAS RN 201998-61-0) was added, followed by DIPEA (1.8 mg, 2.4 μL, 14.1 μmol). After 14 hours DIPEA (3.0 mg, 4.1 μL, 23.5 μmol) was added and the mixture was stirred for additional 24 hours at room temperature. Acetonitrile (0.1 mL) was added and the crude reaction mixture was purified by preparative HPLC to obtain the title compound as purple foam. LC-HRMS (ESI): 919.452 ([M+H]⁺).

### Example 22

### N-[2-[3-[(S or R)-11-1(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-[2-[(4-nitro-2,1,3-benzoxadiazol-7-yl)amino]ethoxy]ethoxy]ethoxy]propanamide

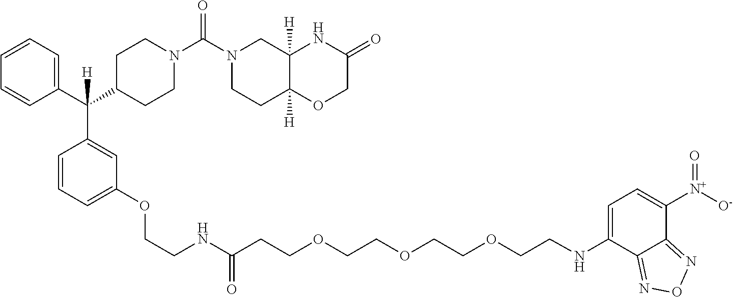 [SMILES: O=C(CCOCCOCCOCCNc1ccc([N+](=O)[O-])c2nonc12)NCCOc1cccc([C@H](c2ccccc2)C2CCN(C(=O)N3CC[C@@H]4OCC(=O)N[C@@H]4C3)CC2)c1]

In analogy to the procedure described in example 1 i), N-[2-[3-[(S or R)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-(2-aminoethoxy)ethoxy]ethoxy]propanamide (example 20) was reacted with 4-chloro-7-nitrobenz-2-oxa-1,3-diazole to obtain the title compound as yellow foam. LC-HRMS (ESI): 859.3996 ([M+H]⁺)

### Example 23

### tert-Butyl (2-(3-((1-((4aR,8aS)-3-oxooctahydro-2H-pyrido[4,3-b][1,4]oxazine-6-carbonyl)piperidin-4-ylidene)(phenyl)methyl)phenoxy)ethyl)carbamate

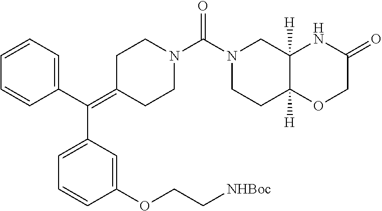 [SMILES: CC(C)(C)OC(=O)NCCOc1cccc(C(=C2CCN(C(=O)N3CC[C@@H]4OCC(=O)N[C@@H]4C3)CC2)c2ccccc2)c1]

### Step a) tert-Butyl (2-(3-(phenyl(piperidin-4-ylidene)methyl)phenoxy)ethyl)carbamate

Pd/C 10% (10%, 10 mg) was added to a solution of benzyl 4-4342-((tert-butoxycarbonyl)amino)ethoxy)phenyl)(phenyl)methylene)piperidine-1-carboxylate (110 mg, 0.2 mmol) in ethanol (10 mL). The reaction vessel was purged with hydrogen gas. The mixture was stirred for 5 hours at room temperature under 1 atm hydrogen gas pressure. The catalyst was filtered off through short plug of celite and washed with ethanol. The filtrate was concentrated in vacuo to provide the title compound (81 mg, quantitative) as yellow oil. NMR of the crude revealed desired product.

### Step b) tert-Butyl (2-(34(1-(3-oxooctahydro-2H-pyrido[4,3-b][1,4]oxazine-6-carbonyl)piperidin-4-ylidene)(phenyl)methyl)phenoxy)ethyl)carbamate

A mixture of (+)-cis-4a,5,6,7,8,8a-hexahydro-4H-pyrido[4,3-b][1,4]oxazin-3-one as a mixture with chiral auxillaries (MW=744 g/mol, 201 mg, 0.27 mmol) and sodium bicarbonate (92 mg, 1.1 mmol) in DCM (4 mL) was cooled to 0° C. Triphosgene (57 mg, 191 μmol) was added and the mixture was stirred at ambient temperature for 14 hours and cooled to 0° C. again. tert-butyl (2-(3-(phenyl(piperidin-4-ylidene)methyl)phenoxy)ethyl)carbamate (115 mg, 0.27 mmol) and DIPEA (142 mg, 191 μL, 1.1 mmol) were added. The suspension was stirred at ambient temperature for 2 hours. EtOAc and H₂O water were added. The layers were separated and the organic layer dried over Na₂SO₄. The solvent was removed under reduced pressure and the crude was purified column chromatography (SiO₂, 30 to 100% EtOAc in cyclohexane, than 0 to 10% MeOH in DCM) to provide the title compound (50 mg, 32%) as white oil. LC-HRMS (ESI): m/z=591.3172 ([M+H]⁺).

### Example 24

### (4aR,8aS)-6-(4-43-(2-((7-Nitrobenzo[c][1,2,5]oxadiazol-4-yl)amino)ethoxy)phenyl)(phenyl)methylene)piperidine-1-carbonyl)hexahydro-2H-pyrido[4,3-b][1,4]oxazin-3(4H)-one

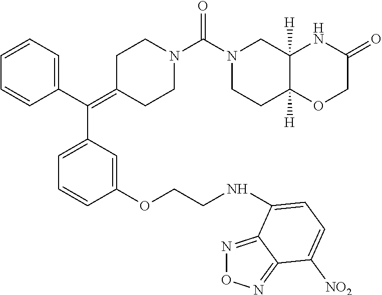 [SMILES: O=C1CO[C@H]2CCN(C(=O)N3CCC(=C(c4ccccc4)c4cccc(OCCNc5ccc([N+](=O)[O-])c6nonc56)c4)CC3)C[C@H]2N1]

### Step a) (4aR,8aS)-6-(4(3-(2-Aminoethoxy)phenyl)(phenyl)methylene)piperidine-1-carbonyl)hexahydro-2H-pyrido[4,3-b][1,4]oxazin-3(4H)-one

In analogy to the procedure described in example 1 h) tert-butyl (2-(3-((1-(3-oxooctahydro-2H-pyrido[4,3-b][1,4]oxazine-6-carbonyl)piperidin-4-ylidene)(phenyl)methyl)phenoxy)ethyl)carbamate was deprotected with TFA to obtain the title compound as off-white solid. LCMS (ESI): 491.1 ([M+H]⁺).

### Step b) 3-(2-(2-(2-((7-Nitrobenzo[c][1,2,5]oxadiazol-4-yl)amino)ethoxy)ethoxy)ethoxy)-N-(2-(3-((1-(4aR,8aS)-3-oxooctahydro-2H-pyrido[4,3-b][1,4]oxazine-6-carbonyl)piperidin-4-yl)(phenyl)methyl)phenoxy)ethyl)propanamide

In analogy to the procedure described in example 1 i), 3-(2-aminoethoxy)-N-(2-(3-((1-((4aR,8aS)-3-oxooctahydro-2H-pyrido[4,3-b][1,4]oxazine-6-carbonyl)piperidin-4-yl)(phenyl)methyl)phenoxy)ethyl)propanamide was reacted with 4-chloro-7-nitrobenz-2-oxa-1,3-diazole to obtain the title compound as orange solid. LC-HRMS (ESI): 654.2675 ([M+H]⁺).

### Example 25

### tert-Butyl (2-(4-((1-((4aR,8aS)-3-oxooctahydro-2H-pyrido[4,3-b][1,4]oxazine-6-carbonyl)piperidin-4-ylidene)(phenyl)methyl)phenoxy)ethyl)carbamate

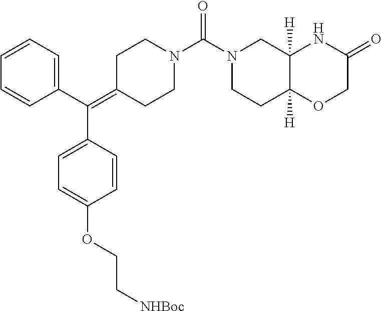 [SMILES: CC(C)(C)OC(=O)NCCOc1ccc(C(=C2CCN(C(=O)N3CC[C@@H]4OCC(=O)N[C@@H]4C3)CC2)c2ccccc2)cc1]

### Step a) Benzyl 4-((4-(2-((tert-butoxycarbonyl)amino)ethoxy)phenyl)(phenyl)methylene)piperidine-1-carboxylate

A mixture of benzyl (Z)-4-(phenyl(2-tosylhydrazineylidene)methyl)piperidine-1-carboxylate (200 mg, 0.4 mmol), tetrakis(triphenylphosphine)palladium(0) (46 mg, 40 μmol), lithium tert-butoxide (48 mg, 0.6 mmol) and N-Boc-2-(4-bromophenoxy)ethylamine (200 mg, 0.6 mmol) in 1,4-dioxane (8 mL) was stirred at 80° C. for 14 hours. EtOAc and H₂O water were added. The layers were separated and the organic layer dried over Na₂SO₄. The solvent was removed under reduced pressure and the crude was purified by flash chromatography (SiO₂, 15 g cartridge, cyclohexane EtOAc 0-30%) to provide the title compound (52 mg, 25%) as light yellow foam. LCMS (ESI): 565.1 ([M+Na]⁺).

### Step b) (4aR,8aS)-6-((4(4-(2-Aminoethoxy)phenyl)(phenyl)methylene)piperidine-1-carbonyl)hexahydro-2H-pyrido[4,3-b][1,4]oxazin-3 (4H)-one

In analogy to the procedure described in example 23a) benzyl 4-4442-((tert-butoxycarbonyl)amino)ethoxy)phenyl)(phenyl)methylene)piperidine-1-carboxylate was deprotected with Pd/C and hydrogen to obtain the title compound as brown oil. LCMS (ESI): 409.1 ([M+H]⁺).

### Step c) tert-Butyl (2-((4-((1(4aR,8aS)-3-oxooctahydro-2H-pyrido[4,3-b][1,4]oxazine-6-carbonyl)piperidin-4-ylidene)(phenyl)methyl)phenoxy)ethyl)carbamate

In analogy to the procedure described in example 23 b) (4aR,8aS)-6-(4-44-(2-aminoethoxy)phenyl)(phenyl)methylene)piperidine-1-carbonyl)hexahydro-2H-pyrido[4,3-b][1,4]oxazin-3(4H)-one formed urea with (+)-cis-4a,5,6,7,8,8a-hexahydro-4H-pyrido[4,3-b][1,4]oxazin-3-one utilizing triphosgene to provide the title compound as transparent oil. LC-HRMS (ESI): 591.3168 ([M+H]⁺).

The following examples can be prepared in analogy to Examples 1 to 25.

### Example 26

### tert-Butyl N-13-13-111-1(4aR,8aS)-3-oxo-4,4a,5,7,8,8a-hexahydropyrido[4,3-b][1,4]oxazine-6-carbonyl]-4-piperidylidene]-phenyl-methyl]phenyl]prop-2-ynyl]carbamate

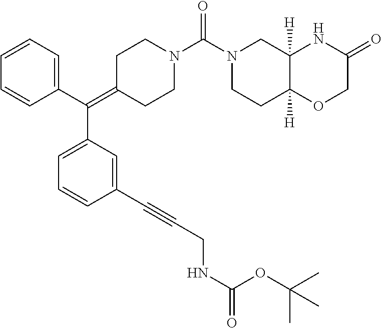 [SMILES: CC(C)(C)OC(=O)NCC#Cc1cccc(C(=C2CCN(C(=O)N3CC[C@@H]4OCC(=O)N[C@@H]4C3)CC2)c2ccccc2)c1]

### Example 27

### tert-Butyl N-13-14-111-1(4aR,8aS)-3-oxo-4,4a,5,7,8,8a-hexahydropyrido[4,3-b][1,4]oxazine-6-carbonyl]-4-piperidylidene]-phenyl-methyl]phenyl]prop-2-ynyl]carbamate

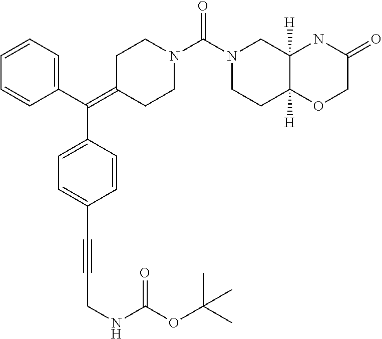 [SMILES: CC(C)(C)OC(=O)NCC#Cc1ccc(C(=C2CCN(C(=O)N3CC[C@@H]4OCC(=O)N[C@@H]4C3)CC2)c2ccccc2)cc1]

### Example 28

### tert-Butyl N-[(E)-3-[3-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenyl]allyl]carbamate

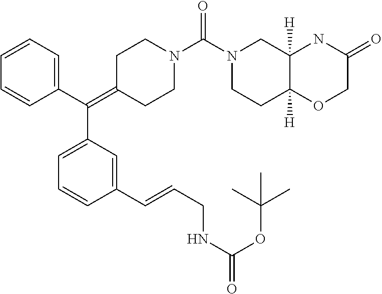 [SMILES: CC(C)(C)OC(=O)NC/C=C/c1cccc(C(=C2CCN(C(=O)N3CC[C@@H]4OCC(=O)N[C@@H]4C3)CC2)c2ccccc2)c1]

### Example 29

### tert-Butyl N-[(E)-3-[4-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenyl]allyl]carbamate

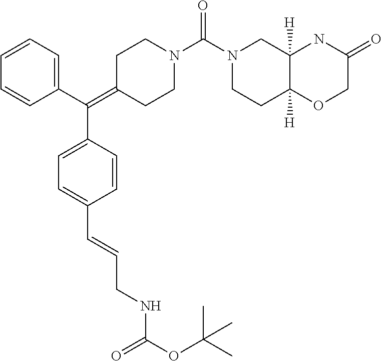 [SMILES: CC(C)(C)OC(=O)NC/C=C/c1ccc(C(=C2CCN(C(=O)N3CC[C@@H]4OCC(=O)N[C@@H]4C3)CC2)c2ccccc2)cc1]

### Example 30

### tert-Butyl N-12-13-[(SR)-11-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate

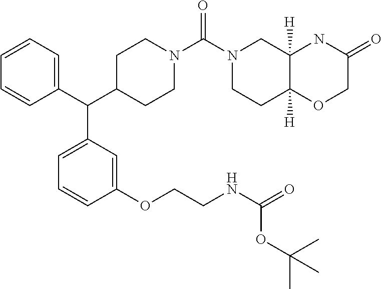 [SMILES: CC(C)(C)OC(=O)NCCOc1cccc(C(c2ccccc2)C2CCN(C(=O)N3CC[C@@H]4OCC(=O)N[C@@H]4C3)CC2)c1]

### Example 31

### tert-Butyl N-[2-[4-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate

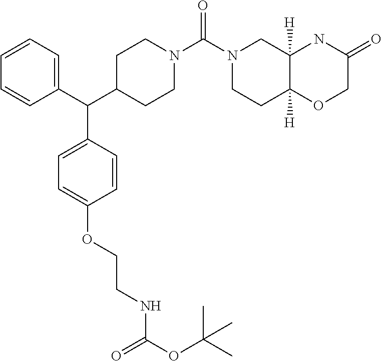 [SMILES: CC(C)(C)OC(=O)NCCOc1ccc(C(c2ccccc2)C2CCN(C(=O)N3CC[C@@H]4OCC(=O)N[C@@H]4C3)CC2)cc1]

### Example 32

### tert-Butyl N-[3-[3-(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenyl]propyl]carbamate

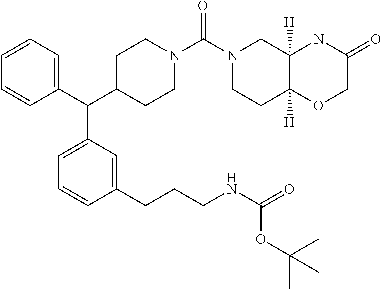 [SMILES: CC(C)(C)OC(=O)NCCCc1cccc(C(c2ccccc2)C2CCN(C(=O)N3CC[C@@H]4OCC(=O)N[C@@H]4C3)CC2)c1]

### Example 33

### tert-Butyl N-[3-[4-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenyl]propyl]carbamate

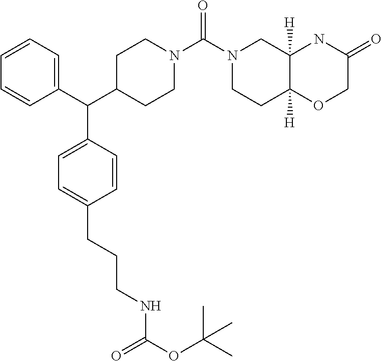 [SMILES: CC(C)(C)OC(=O)NCCCc1ccc(C(c2ccccc2)C2CCN(C(=O)N3CC[C@@H]4OCC(=O)N[C@@H]4C3)CC2)cc1]

### Example 34

### tert-Butyl N-[3-[4-[(SR)-[1-1(4aR,8aS)-3-oxo-4,4a,5,7,8,8a-hexahydropyrido[4,3-b][1,4]oxazine-6-carbonyl]-4-piperidyl]-phenyl-methyl]phenyl]prop-2-ynyl]carbamate

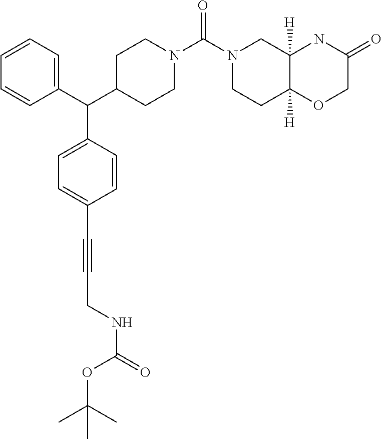 [SMILES: CC(C)(C)OC(=O)NCC#Cc1ccc(C(c2ccccc2)C2CCN(C(=O)N3CC[C@@H]4OCC(=O)N[C@@H]4C3)CC2)cc1]

### Example 35

### tert-Butyl N-[3-[3-[(SR)-[1-1(4aR,8aS)-3-oxo-4,4a,5,7,8,8a-hexahydropyrido[4,3-b][1,4]oxazine-6-carbonyl]-4-piperidyl]-phenyl-methyl]phenyl]prop-2-ynyl]carbamate

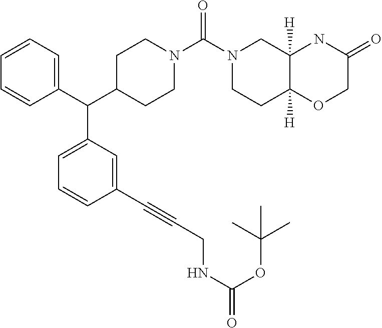 [SMILES: CC(C)(C)OC(=O)NCC#Cc1cccc(C(c2ccccc2)C2CCN(C(=O)N3CC[C@@H]4OCC(=O)N[C@@H]4C3)CC2)c1]

### Example 36

### tert-Butyl N-[(E)-3-[1-[(SR)-[1-1(4aR,8aS)-3-oxo-4,4a,5,7,8,8a-hexahydropyrido[4,3-b][1,4]oxazine-6-carbonyl]-4-piperidyl]-phenyl-methyl]phenyl]allyl]carbamate

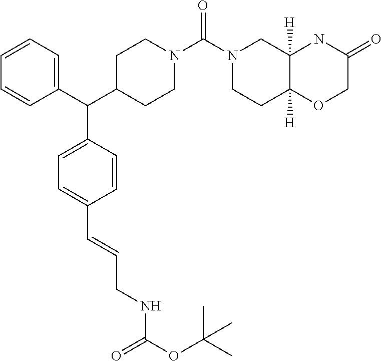 [SMILES: CC(C)(C)OC(=O)NC/C=C/c1ccc(C(c2ccccc2)C2CCN(C(=O)N3CC[C@@H]4OCC(=O)N[C@@H]4C3)CC2)cc1]

### Example 37

### tert-Butyl N-[(E)-3-[3-[(SR)-[1-1(4aR,8aS)-3-oxo-4,4a,5,7,8,8a-hexahydropyrido[4,3-b][1,4]oxazine-6-carbonyl]-4-piperidyl]-phenyl-methyl]phenyl]allyl]carbamate

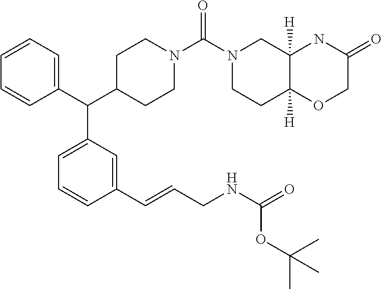 [SMILES: CC(C)(C)OC(=O)NC/C=C/c1cccc(C(c2ccccc2)C2CCN(C(=O)N3CC[C@@H]4OCC(=O)N[C@@H]4C3)CC2)c1]

### Example 38

### tert-Butyl N-[2-[3-[2-[3-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenoxy]ethylamino]-3-oxo-propoxy]ethyl]carbamate

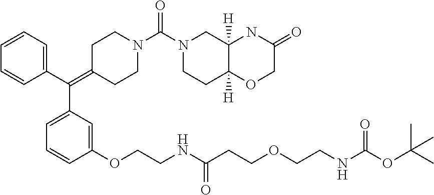 [SMILES: CC(C)(C)OC(=O)NCCOCCC(=O)NCCOc1cccc(C(=C2CCN(C(=O)N3CC[C@@H]4OCC(=O)N[C@@H]4C3)CC2)c2ccccc2)c1]

### Example 39

### tert-Butyl N-[3-[4-[[1-1(4aR,8aS)-3-oxo-4,4a,5,7,8,8a-hexahydropyrido[4,3-b][1,4]oxazine-6-carbonyl]-4-piperidylidene]-phenyl-methyl]phenyl]propyl]carbamate

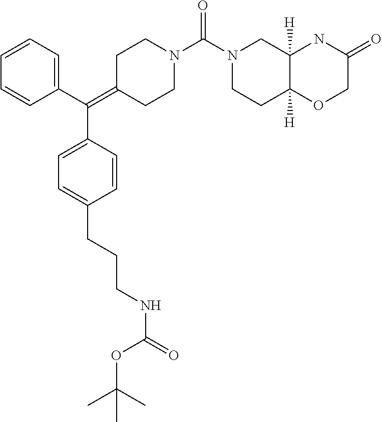 [SMILES: CC(C)(C)OC(=O)NCCCc1ccc(C(=C2CCN(C(=O)N3CC[C@@H]4OCC(=O)N[C@@H]4C3)CC2)c2ccccc2)cc1]

### Example 40

### tert-Butyl N-[3-[3-[[1-1(4aR,8aS)-3-oxo-4,4a,5,7,8,8a-hexahydropyrido[4,3-b][1,4]oxazine-6-carbonyl]-4-piperidylidene]-phenyl-methyl]phenyl]propyl]carbamate

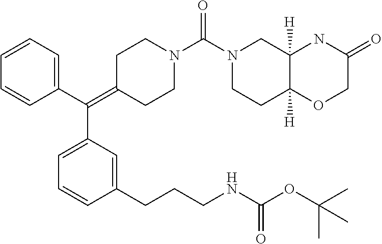 [SMILES: CC(C)(C)OC(=O)NCCCc1cccc(C(=C2CCN(C(=O)N3CC[C@@H]4OCC(=O)N[C@@H]4C3)CC2)c2ccccc2)c1]

1. A compound of formula (I) 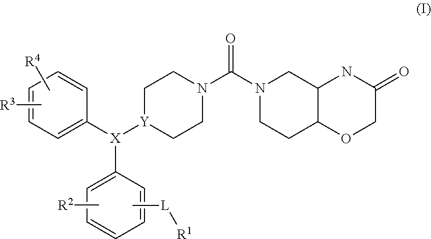
or a pharmaceutically acceptable salt thereof, wherein:
L is a linker selected from 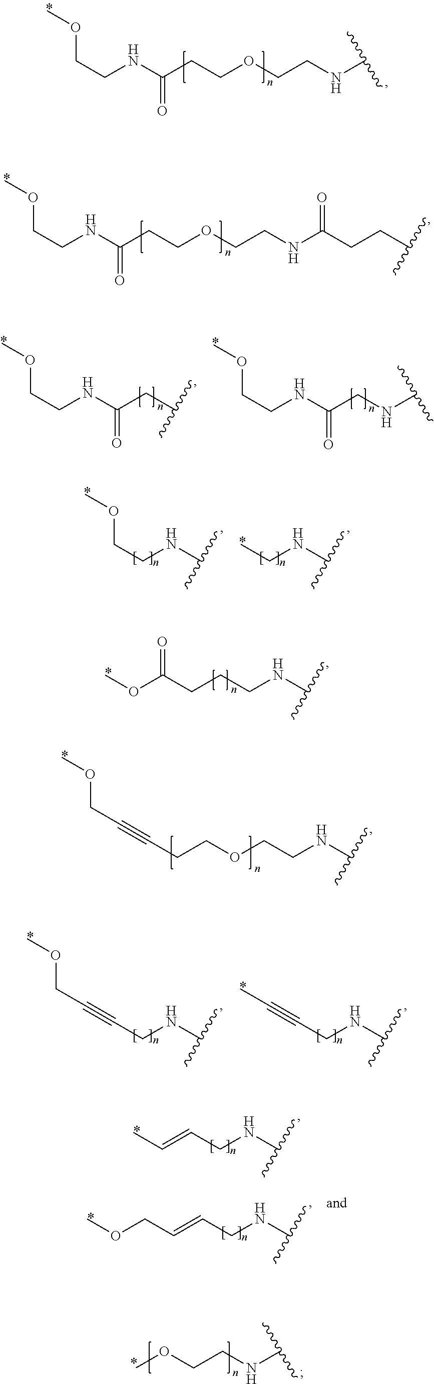
wherein
each occurrence of n is independently an integer selected from 1, 2, 3, 4, 5, 6, and 7;
a wavy line indicates the point of attachment to R¹; and
an asterisk indicates the point of attachment to the rest of formula (I);
R¹ is a fluorescent label selected from 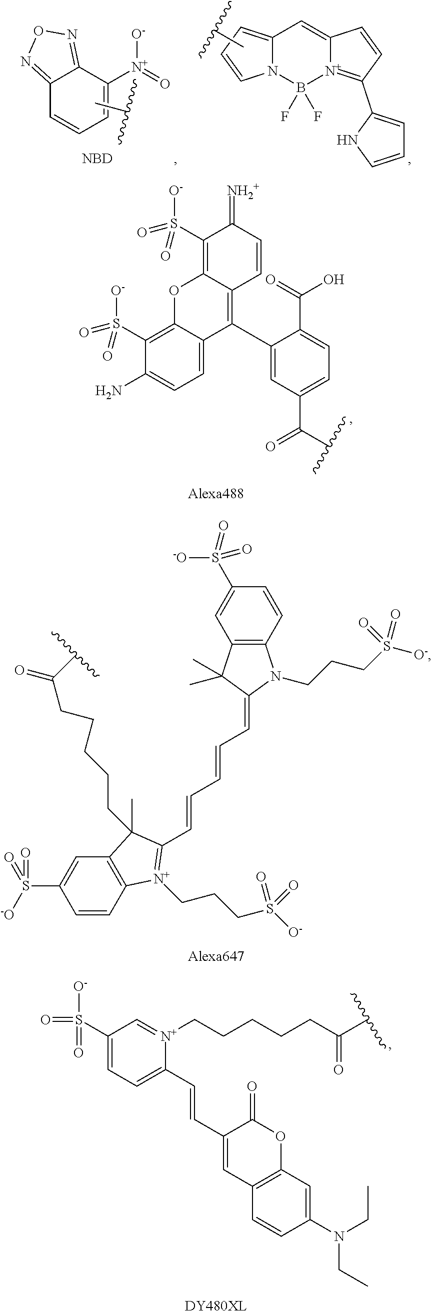 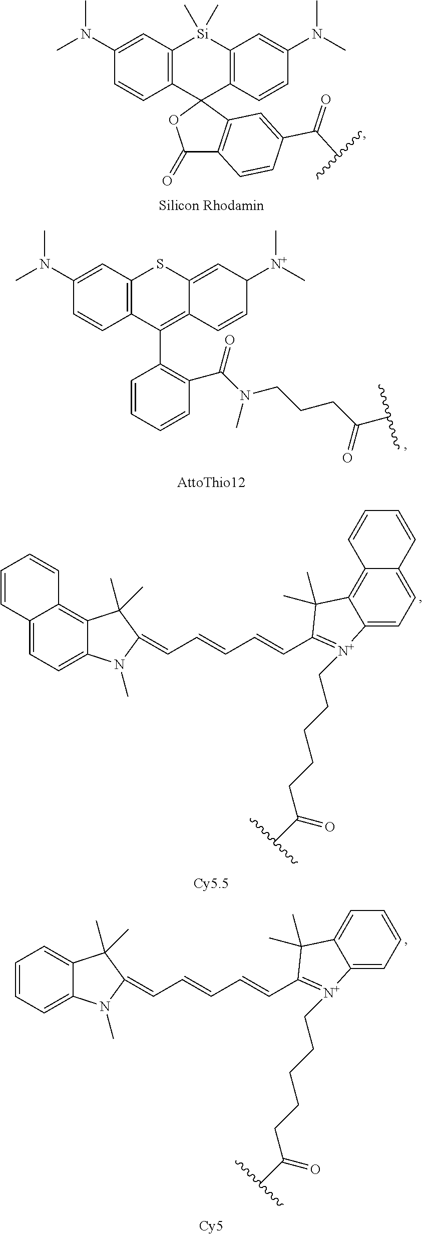 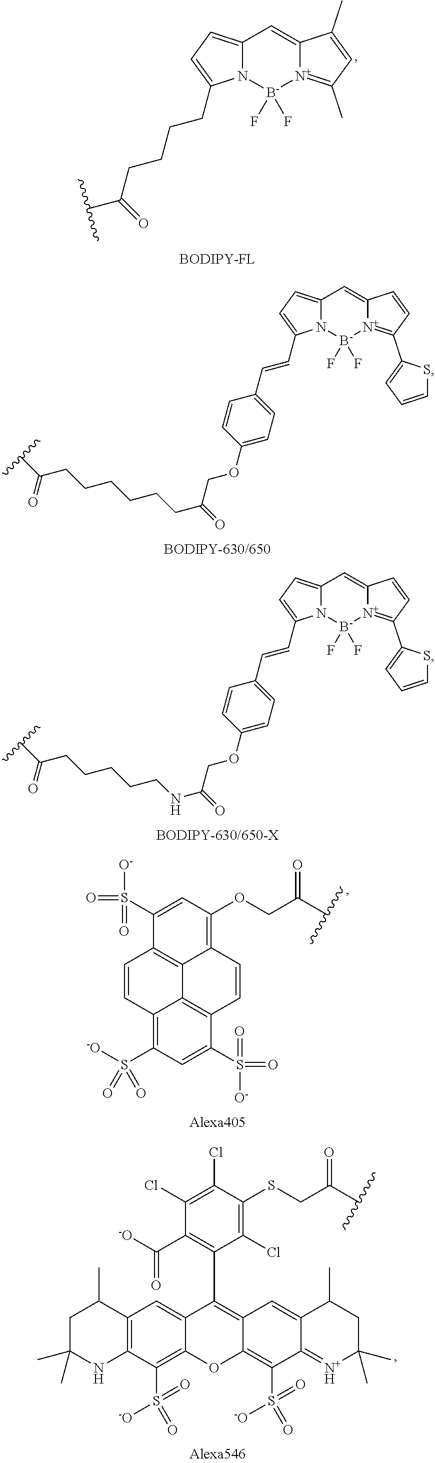 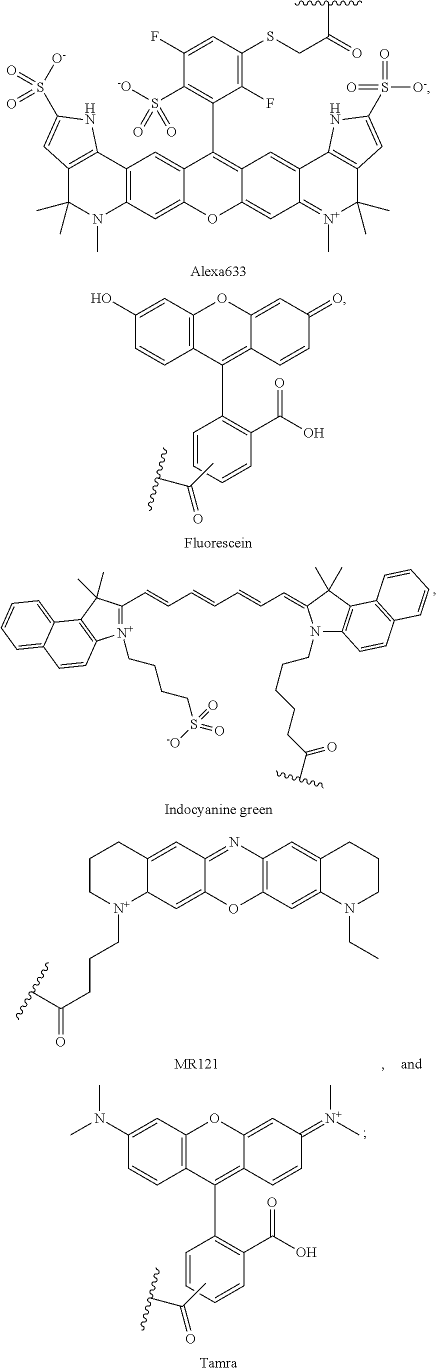
wherein a wavy line indicates the point of attachment to the linker L;
R², R³, and R⁴ are each independently selected from hydrogen, halogen, C₁-C₆-alkyl, halo-C₁-C₆-alkyl, C₁-C₆-alkoxy and halo-C₁-C₆-alkoxy; and
X and Y are both CH; or
X and Y taken together form a double bond (C═C).

2. The compound of formula (I) according to claim 1, or a pharmaceutically acceptable salt thereof, wherein the linker L is selected from 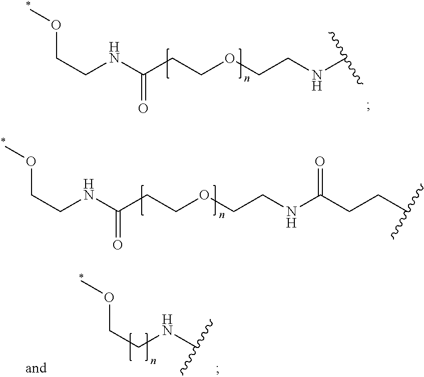
wherein
each occurrence of n is independently an integer selected from 1, 2, and 3;
a wavy line indicates the point of attachment to the fluorescent label R¹; and
an asterisk indicates the point of attachment to the rest of formula (I).

3. The compound of formula (I) according to claim 2, or a pharmaceutically acceptable salt thereof, wherein the linker L is selected from 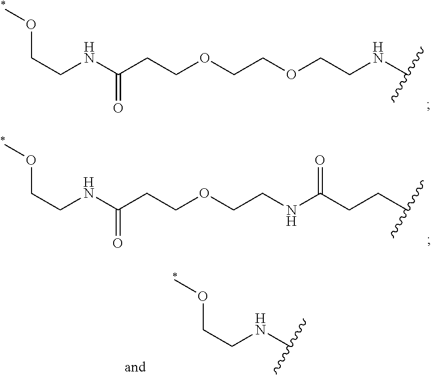 [SMILES: *NCCOCCOCCC(=O)NCCO* ; *CCC(=O)NCCOCCC(=O)NCCO* ; *NCCO*]
wherein
a wavy line indicates the point of attachment to the fluorescent label R¹; and
an asterisk indicates the point of attachment to the rest of formula (I).

4. The compound of formula (I) according to any one of claims 1 to 3, or a pharmaceutically acceptable salt thereof, wherein the fluorescent label R¹ is selected from 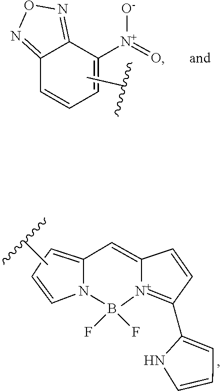
wherein a wavy line indicates the point of attachment to the linker L.

5. The compound of formula (I) according to any one of claims 1 to 4, or a pharmaceutically acceptable salt thereof, wherein R² is hydrogen.

6. The compound of formula (I) according to any one of claims 1 to 5, or a pharmaceutically acceptable salt thereof, wherein R³ is hydrogen.

7. The compound of formula (I) according to any one of claims 1 to 6, or a pharmaceutically acceptable salt thereof, wherein R⁴ is hydrogen.

8. The compound of formula (I) according to claim 1, or a pharmaceutically acceptable salt thereof, wherein the compound of formula (I) is a compound of formula (II) or (III) 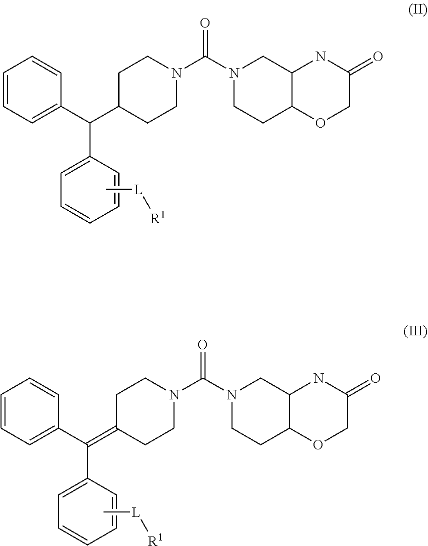
wherein the linker L is selected from 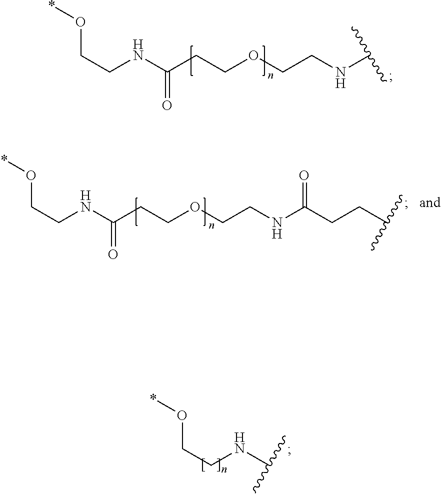
wherein
each occurrence of n is independently an integer selected from 1, 2, and 3;
a wavy line indicates the point of attachment to the fluorescent label R¹; and
an asterisk indicates the point of attachment to the rest of formula (I);
and wherein the fluorescent label R¹ is selected from 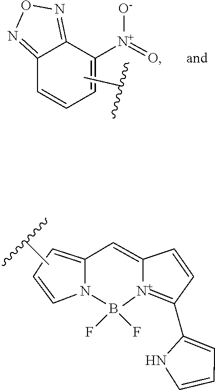
wherein a wavy line indicates the point of attachment to the linker L.

9. The compound of formula (I) according to claim 8, or a pharmaceutically acceptable salt thereof, wherein:
the linker L is selected from 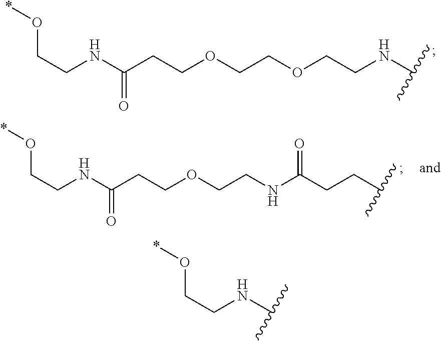 [SMILES: *NCCOCCOCCC(=O)NCCO* ; *CCC(=O)NCCOCCC(=O)NCCO* ; *NCCO*]
wherein
a wavy line indicates the point of attachment to the fluorescent label R¹; and
an asterisk indicates the point of attachment to the rest of formula (I);
and wherein the fluorescent label R¹ is selected from 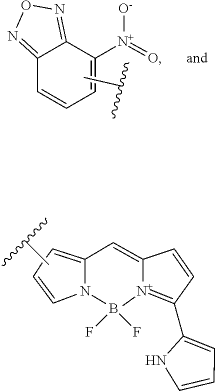
wherein a wavy line indicates the point of attachment to the linker L.

10. The compound of formula (I) according to any one of claims 1 to 9, or a pharmaceutically acceptable salt thereof, wherein the compound of formula (I) is selected from:
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[(4-nitro-2,1,3-benzoxadiazol-7-yl)amino]ethoxy]propanamide;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[(4-nitro-2,1,3-benzoxadiazol-7-yl)amino]ethoxy]propanamide;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-[(4-nitro-2,1,3-benzoxadiazol-7-yl)amino]ethoxy]ethoxy]propanamide;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-[(4-nitro-2,1,3-benzoxadiazol-7-yl)amino]ethoxy]ethoxy]propanamide;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-[(4-nitro-2,1,3-benzoxadiazol-7-yl)amino]ethoxy]ethoxy]propanamide;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[(4-nitro-2,1,3-benzoxadiazol-7-yl)amino]ethoxy]propanamide;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-[24(4-nitro-2,1,3-benzoxadiazol-7-yl)amino]ethoxy]ethoxy]ethoxy]propanamide;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-[2-[(4-nitro-2,1,3-benzoxadiazol-7-yl)amino]ethoxy]ethoxy]ethoxy]propanamide;
N-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethyl]-3-[2,2-difluoro-12-(1H-pyrrol-2-yl)-3-aza-1-azonia-2-boranuidatricyclo[7.3.0.03,7]dodeca-1(12),4,6,8,10-pentaen-4-yl]propanamide; and
(4aR,8a5)-6-(4-((3-(2-((7-Nitrobenzo[c][1,2,5]oxadiazol-4-yl)amino)ethoxy)phenyl)(phenyl)methylene)piperidine-1-carbonyl)hexahydro-2H-pyrido[4,3-b][1,4]oxazin-3(4H)-one.

11. The compound of formula (I) according to claim 10, or a pharmaceutically acceptable salt thereof, wherein the compound of formula (I) is selected from:
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-[(4-nitro-2,1,3-benzoxadiazol-7-yl)amino]ethoxy]ethoxy]propanamide;
N-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethyl]-3-[2,2-difluoro-12-(1H-pyrrol-2-yl)-3-aza-1-azonia-2-boranuidatricyclo[7.3.0.03,7]dodeca-1(12),4,6,8,10-pentaen-4-yl]propanamide; and
(4aR,8a5)-6-(4-((3-(2-((7-Nitrobenzo[c][1,2,5]oxadiazol-4-yl)amino)ethoxy)phenyl)(phenyl)methylene)piperidine-1-carbonyl)hexahydro-2H-pyrido[4,3-b][1,4]oxazin-3(4H)-one.

12. A compound selected from:
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-(2-aminoethoxy)propanamide;
tert-butyl N-[2-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethoxy]ethyl]carbamate;
tert-butyl N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-[2-(2-aminoethoxy)ethoxy]ethoxy]propanamide;
tert-butyl N-[2-[2-[2-[3-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethylamino]-3-oxo-propoxy]ethoxy]ethoxy]ethyl]carbamate;
N-[2-[3-[[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]-3-[2-(2-aminoethoxy)ethoxy]propanamide;
tert-Butyl (2-(3-((1-((4aR,8a5)-3-oxooctahydro-2H-pyrido[4,3-b][1,4]oxazine-6-carbonyl)piperidin-4-ylidene)(phenyl)methyl)phenoxy)ethyl)carbamate;
tert-Butyl (2-(4-((1-((4aR,8a5)-3-oxooctahydro-2H-pyrido[4,3-b][1,4]oxazine-6-carbonyl)piperidin-4-ylidene)(phenyl)methyl)phenoxy)ethyl)carbamate;
tert-Butyl N-[3-[3-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenyl]prop-2-ynyl]carbamate;
tert-Butyl N-[3-[4-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenyl]prop-2-ynyl]carbamate;
tert-Butyl N-[(E)-[3-[4-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenyl]allyl]carbamate;
tert-Butyl N-(E)-3-[4-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenyl]allyl]carbamate;
tert-Butyl N-[2-[3-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate;
tert-Butyl N-[2-[4-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenoxy]ethyl]carbamate;
tert-Butyl N-[3-[3-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenyl]propyl]carbamate;
tert-Butyl N-[3-[4-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenyl]propyl]carbamate;
tert-Butyl N4344-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenyl]prop-2-ynyl]carbamate;
tert-Butyl N-[3-[3-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenyl]prop-2-ynyl]carbamate;
tert-Butyl N-[(E)-3-[4-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenyl]allyl]carbamate;
tert-Butyl N-(E)-343-[(SR)-[1-[(4aR,8aS)-3-oxo-4,4a,5,7,8,8a-hexahydropyrido[4,3-b][1,4]oxazine-6-carbonyl]-4-piperidyl]-phenyl-methyl]phenyl]allyl]carbamate;
tert-Butyl N42434243-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenoxy]ethylamino]-3-oxo-propoxy]ethyl]carbamate;
tert-Butyl N-[3-[4-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenyl]propyl]carbamate;
tert-Butyl N-[3-[3-[[1-[(4aR,8aS)-3-oxo-4,4a,5,7,8,8a-hexahydropyrido[4,3-b][1,4]oxazine-6-carbonyl]-4-piperidylidene]-phenyl-methyl]phenyl]propyl]carbamate.

13. Use of a compound according to claim 12, for the preparation of a compound according to any one of claims 1 to 11.

14. A process of manufacturing the compounds of formula (I) according to any one of claims 1 to 11, comprising:
(a) reacting a first amine 4a,5,6,7,8,8a-hexahydro-4H-pyrido[4,3-b][1,4]oxazin-3-one (2), 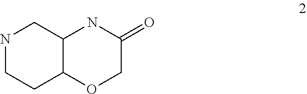 [SMILES: O=C1COC2CCNCC2N1]
with a second amine 1, wherein R², R³, R⁴, and L are as defined in any one of claims 1 to 11, and PG is a suitable protective group, 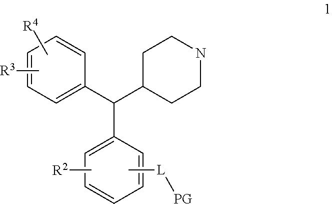
in the presence of a base and a urea forming reagent,
to form a compound of formula 3, wherein R², R³, R⁴, and L are as defined in any one of claims 1 to 11, and PG is a suitable protective group 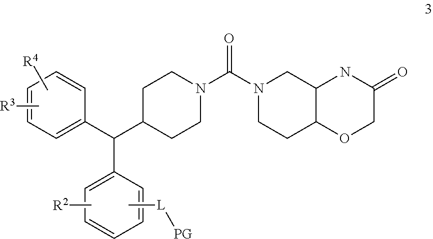
or
(b) reacting a first amine 4a,5,6,7,8,8a-hexahydro-4H-pyrido[4,3-b][1,4]oxazin-3-one (2), 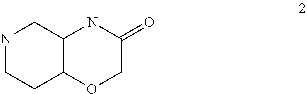 [SMILES: O=C1COC2CCNCC2N1]
with a second amine 1a, wherein R¹, R², R³, R⁴, and L are as defined in any one of claims 1 to 11 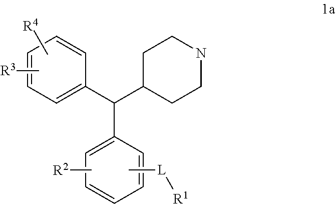
in the presence of a base and a urea forming reagent,
to form said compound of formula (I).

15. A compound of formula (I) according to any one of claims 1 to 11, when manufactured according to the process of claim 14.

16. A compound of formula (I) according to any one of claims 1 to 11, for use in monoacylglycerol lipase (MAGL) occupancy studies.

17. A compound of formula (I) according to any one of claims 1 to 11, for use in diagnostic imaging of monoacylglycerol lipase (MAGL) in a mammal.

18. A compound of formula (I) according to any one of claims 1 to 11, for use in generating monoacylglycerol lipase (MAGL) equilibrium and kinetic binding data.

19. Use of a compound of formula (I) according to any one of claims 1 to 11 in monoacylglycerol lipase (MAGL) occupancy studies.

20. Use of a compound of formula (I) according to any one of claims 1 to 11 in diagnostic imaging of monoacylglycerol lipase (MAGL) in a mammal.

21. Use of a compound of formula (I) according to any one of claims 1 to 11 for generating monoacylglycerol lipase (MAGL) equilibrium and kinetic binding data.

22. A method of studying monoacylglycerol lipase (MAGL) occupancy, comprising contacting MAGL with a compound of formula (I) according to any one of claims 1 to 11.

23. A method of diagnostic imaging of monoacylglycerol lipase (MAGL) in a mammal, comprising contacting MAGL with a compound of formula (I) according to any one of claims 1 to 11.

24. A method of generating monoacylglycerol lipase (MAGL) equilibrium and kinetic binding data, comprising contacting MAGL with a compound of formula (I) according to any one of claims 1 to 11.

25. The invention as described hereinbefore.
